SEQUENCE LISTING (7 sequences)

SEQ ID NO: 1 - protein, 17 aa, Homo sapiens
  MISC_FEATURE (2)..(2): Xaa at position 2 may be Asn, His or Tyr
  MISC_FEATURE (4)..(4): Xaa at position 4 may be Gly, Ser, Thr or Asn
  MISC_FEATURE (8)..(8): Xaa at position 8 may be Thr or Arg
  MISC_FEATURE (15)..(15): Xaa at position 15 may be Ala, Asp, Glu, Gly or Ser
  MISC_FEATURE (16)..(16): Xaa at position 16 may be Ser or Thr
  MISC_FEATURE (17)..(17): Xaa at position 17 may be Asp or Glu
CXCXGPPXFECWCYXXX

SEQ ID NO: 2 - protein, 18 aa, Homo sapiens
  MISC_FEATURE (3)..(3): Xaa at position 3 may be Asn, His or Tyr
  MISC_FEATURE (5)..(5): Xaa at position 5 may be Gly, Ser, Thr or Asn
  MISC_FEATURE (9)..(9): Xaa at position 9 may be Thr or Arg
  MISC_FEATURE (16)..(16): Xaa at position 16 may be Ala, Asp, Glu, Gly or Ser
  MISC_FEATURE (17)..(17): Xaa at position 17 may be Ser or Thr
  MISC_FEATURE (18)..(18): Xaa at position 18 may be Asp or Glu
SCXCXGPPXFECWCYXXX

SEQ ID NO: 3 - protein, 22 aa, Homo sapiens
  MISC_FEATURE (5)..(5): Xaa at position 5 may be Asn, His or Tyr
  MISC_FEATURE (7)..(7): Xaa at position 7 may be Gly, Ser, Thr or Asn
  MISC_FEATURE (11)..(11): Xaa at position 11 may be Thr or Arg
  MISC_FEATURE (18)..(18): Xaa at position 18 may be Ala, Asp, Glu, Gly or Ser
  MISC_FEATURE (19)..(19): Xaa at position 19 may be Ser or Thr
  MISC_FEATURE (20)..(20): Xaa at position 20 may be Asp or Glu
AGSCXCXGPPXFECWCYXXXGT

SEQ ID NO: 4 - protein, 4 aa, Homo sapiens
GGGK

SEQ ID NO: 5 - protein, 4 aa, Homo sapiens
GSGK

SEQ ID NO: 6 - protein, 5 aa, Homo sapiens
GSGSK

SEQ ID NO: 7 - protein, 26 aa, Homo sapiens
AGSCYCSGPPRFECWCYETEGTGGGK

(19) United States
(12) Patent Application Publication
(10) Pub. No.: US 2022/0273830 A1
(43) Pub. Date: Sep. 1, 2022

(54) COMPOUNDS AND METHODS OF USE

(71) Applicant: EDINBURGH MOLECULAR IMAGING LIMITED, Edinburgh (GB)

(72) Inventor: Christophe Frederic Portal, Edinburgh (GB)

(21) Appl. No.: 17/618,850

(22) PCT Filed: Jun. 15, 2020
(86) PCT No.: PCT/GB2020/051442

§ 371 Date: Dec. 13, 2021

(30) Foreign Application Priority Data
Jun. 14, 2019 (GB) 1908573.7
Mar. 26, 2020 (GB) 2004360.0

(51) Int. Cl. A61K 51/08; A61P 35/00

(52) U.S. Cl. CPC A61K 51/088; A61P 35/00

(57) ABSTRACT

There is described compounds, in particular compounds comprising a cMet binding cyclic peptide for human or animal administration and methods of use thereof. In particular, there is described compounds suitable for the preparation of an agent for use in imaging and/or radiotherapy. Also described are a pharmaceutical composition and a kit for the preparation of the pharmaceutical composition. There are also described methods of imaging using the compound or pharmaceutical composition, such as in detection, diagnosis, prognosis, prediction of outcome, surgery, staging, treatment, therapy, radiotherapy, monitoring of treatment, monitoring of disease progression and/or monitoring therapy of conditions such as cancer. Further described is the use of the compound or pharmaceutical composition as at least one of, or both, an imaging agent and a radiotherapeutic agent.

### FIELD OF INVENTION

The present invention relates to compounds for human or animal administration and to methods of use thereof. In particular, the present invention relates to compounds suitable for the preparation of agents for use in imaging that uses radionuclides (i.e., endoradiology or nuclear medical imaging) and/or radiotherapy. Examples of such imaging techniques are single-photon emission computed tomography (SPECT) scintigraphy and positron emission tomography (PET). Examples of radionuclides used in radiotherapy include α-particle emitters, β-particle emitters and Auger electron emitters. Furthermore, the present invention relates to kits and imaging/radiotherapy agents for use in nuclear medical imaging and/or radiotherapy.

### BACKGROUND

Radiolabelled compounds are useful in both molecular imaging and radiotherapy, and several of these have been described in the prior art.

WO2012/022676 describes radiolabelled cMet binding peptides suitable for PET imaging in vivo. The cMet binding peptides are labelled with the radioisotope ¹⁸F. Also described are pharmaceutical compositions, methods of preparation of the agents and compositions, and methods of in vivo imaging using the compositions, especially for use in the management of cancer.

WO2012/119937 describes technetium imaging agents comprising radiolabelled cMet binding peptides suitable for SPECT or PET imaging in vivo. The cMet binding peptides are labelled via chelator conjugates. Also described are pharmaceutical compositions, methods of preparation of the agents and compositions, and methods of in vivo imaging using the compositions, especially for use in the diagnosis of cancer.

WO2005/030266 discloses cMet as a preferred biological target for contrast agents for optical imaging, specifically for colorectal cancer (CRC) diagnosis. WO2005/030266 discloses optical imaging agents which comprise a vector, which has affinity to the abnormally expressed target, a linker moiety and one or more reporter moieties detectable in optical imaging.

WO2004/078778 discloses polypeptides or multimeric peptide constructs which bind cMet or a complex comprising cMet and HGF. WO2004/078778 discloses that the peptides can be labelled with a detectable label for in vitro and in vivo applications, or with a drug for therapeutic applications.

Arulappu et al. (Nucl. Med. 2016, 57, 765-770) discloses a ¹⁸F PET agent developed for cancer imaging of head and neck carcinomas. The agent is based on a 26-amino acid peptide with two internal disulphide bridges, specifically binding to cMet receptor. The lysine residue on the peptide is labelled with ¹⁸F.

However, the synthesis of agents such as this, where the radionuclide is introduced covalently is complex, yields are low and purification steps are required to afford the imaging agent in sufficient purity. In addition, such syntheses require the use of prosthetic groups such as p-^{18F} benzaldehyde as a starting material, which needs to be prepared from ¹⁸F-fluoride. Therefore, similarly to the vast majority of ¹⁸F based PET agents, the preparation requires skilled radiochemists and dedicated equipment such as specific robots and cassettes and shielded “hot” cells. As such, specialist sites are required to implement the synthesis of this particular type of ¹⁸F PET imaging agent.

Due to the problems associated with preparing agents with covalently bound ¹⁸F, the use of alternative radionuclides that can be bound by chelating agents has emerged. For example, ⁶⁷Ga for SPECT imaging and ⁶⁸Ga for PET imaging has become more prevalent, as has the use of Al¹⁸F salts.

Whilst examples of imaging agents comprising a targeting moiety conjugated to a chelator (capable of chelating radioactive isotopes) exist, it would be of benefit to have an imaging and/or radiotherapeutic agent that can target cell-surface cMet overexpression with high affinity binding, favourable kinetic profile, specific uptake and/or rapid systemic clearance, as this would enable diagnostic imaging soon (perhaps as early as 1 hour) after administration and/or would enable more targeted radiotherapy (which reduces toxicity caused by off-target exposure).

Therefore, it is an object of the present invention to obviate or mitigate at least some of the disadvantages of the prior art.

A further object of the invention is to provide a radioimaging and/or radiotherapeutic agent for use in targeting sites of cMet overexpression and/or associated conditions.

### DISCLOSURE OF INVENTION

According to the first aspect of the invention there is provided a compound suitable for the preparation of an agent for use in imaging and/or radiotherapy, the compound having Formula I:

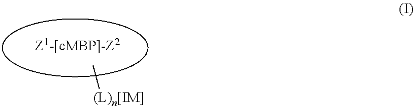

wherein:

Z¹ is attached to the N-terminus of cMBP, and is H or Q;

Z² is attached to the C-terminus of cMBP, and is OH, OB^{C} or Q;
wherein B^{C} is a biocompatible cation;

cMBP is a cMet binding cyclic peptide of 17 to 30 amino acids, which comprises the amino acid sequence (SEQ-1):

Cys^{a}-Xaa¹-Cys^{c}-Xaa²-Gly-Pro-Pro-Xaa³-Phe-Glu-Cys^{d}-Trp-Cys^{b}-Tyr-Xaa⁴-Xaa⁵-Xaa⁶;
wherein: Xaa¹ is Asn, His or Tyr;
   Xaa² is Gly, Ser, Thr or Asn;
   Xaa³ is Thr or Arg;
   Xaa⁴ is Ala, Asp, Glu, Gly or Ser;
   Xaa⁵ is Ser or Thr;
   Xaa⁶ is Asp or Glu;
and Cys^{a-d} are each cysteine residues such that residues a and b as well as c and d are cyclised to form two separate disulphide bonds;

each occurrence of Q is independently at least one of:
a metabolism inhibiting group (M^{IG}), which is a biocompatible group that inhibits or suppresses in vivo metabolism of the peptide,
a tumour retention group (T^{RG}), which is a biocompatible group that enhances retention in tumour cells or the like in vivo, and
   a biodistribution enhancement group (D^{EG}), which is a biocompatible group that enhances biodistribution and/or prolongs blood retention in vivo;
L is a synthetic linker group of formula —(A)ₘ— wherein each A is independently —CR₂—, —CR═CR—, —C═C—, —CR₂CO₂—, —CO₂CR₂—, —NRCO—, —CONR—, —NR(C═O)NR—, —NR(C═S)NR—, —SO₂NR—, —NRSO₂—, —CR₂OCR₂—, —CR₂SCR₂—, CR₂NRCR₂—, a C₄₋₈ cycloheteroalkylene group, a C₄₋₈ cycloalkylene group, a C₅₋₁₂ arylene group, a C₃₋₁₂ heteroarylene group, an amino acid, a sugar or a monodisperse polyethyleneglycol (PEG) building block;
each R is independently chosen from H, C₁₋₄ alkyl, C₂₋₄ alkenyl, C₂₋₄ alkynyl, C₁₋₄ alkoxyalkyl or C₁₋₄ hydroxyalkyl;
m is an integer of value 1 to 20;
n is an integer of value 0 or 1;
IM is a chelating agent suitable for complexing a radioactive moiety.

The compound may comprise the radioactive moiety.

The Z¹ group substitutes the amine group of the last amino acid residue. Thus, when Z¹ is H, the amino terminus of the cMBP terminates in a free NH₂ group of the last amino acid residue. The Z² group substitutes the carbonyl group of the last amino acid residue. Thus, when Z² is OH, the carboxy terminus of the cMBP terminates in the free CO₂H group of the last amino acid residue, and when Z² is OB^{C} that terminal carboxy group is ionised as a CO₂B^{c} group.

By the term “metabolism inhibiting group” (M^{IG}) is meant a biocompatible group which inhibits or suppresses in vivo metabolism of the cMBP peptide at either the amino terminus (Z¹) or carboxy terminus (Z²). Such groups are well known to those skilled in the art and are suitably chosen from, for the peptide amine terminus: N-acylated groups —NH(C═O)RG where the acyl group —(C═O)RG has RG chosen from: C₁₋₆alkyl, C₃₋₁₀ aryl groups or comprises a polyethyleneglycol (PEG) building block. Suitable PEG groups are described for the linker group (L), below. Such PEG groups may be the biomodifiers of Formula IA or IB. Such amino terminus M^{IG} groups may be acetyl, benzyloxycarbonyl or trifluoroacetyl, typically acetyl.

Suitable metabolism inhibiting groups for the peptide carboxyl terminus include: carboxamide, tert-butyl ester, benzyl ester, cyclohexyl ester, amino alcohol or a polyethyleneglycol (PEG) building block. A suitable M group for the carboxy terminal amino acid residue of the cMBP peptide is where the terminal amine of the amino acid residue is N-alkylated with a C₁₋₄ alkyl group, optionally a methyl group. Such M^{IG} groups may be carboxamide or PEG, and typically are carboxamide.

Formula I denotes that the —(L)ₙ[IM] moiety can be attached at any suitable position of Z¹, Z² or cMBP. For Z¹ or Z², the —(L)ₙ[IM] moiety may either be attached to the M^{IG} group when either of Z¹/Z² is a M^{IG}. When Z¹ is H or Z² is OH, attachment of the —(L)ₙ[IM] moiety at the Z¹ or Z² position gives compounds of formulae [IM]—(L)ₙ—[cMBP]-Z² or Z¹—[cMBP]—(L)ₙ—[IM] respectively. Inhibition of metabolism of the cMBP at either peptide terminus may also be achieved by attachment of the —(L)ₙ[IM] moiety in this way, but —(L)ₙ[IM] is outside the definition of M^{IG} herein.

The —(L)ₙ- moiety of Formula I may be attached at any suitable position of the IM. The —(L)ₙ— moiety either takes the place of an existing substituent of the IM, or is covalently attached to the existing substituent of the IM. The —(L)ₙ— moiety is optionally attached via a carboxyalkyl substituent of the IM.

By the term “tumour retention group” (T^{RG}) is meant a biocompatible group which that enhances or improves retention of the compound in tumour cells or the like in vivo. Such groups can be moieties that add to the overall size of the compound and are suitably chosen from, for the peptide amine terminus or the peptide carboxyl terminus: polyamines, polylysines, PEG (monodisperse), albumin, fatty linear carbon chains, sugars (glycosylation).

By the term “biodistribution enhancement group” (D^{EG}) is meant a biocompatible group which enhances biodistribution or prolongs blood retention (enhancing or improving the pharmacokinetics) of the compound in vivo. Such groups are suitably chosen from, for the peptide amine terminus or the peptide carboxyl terminus: polyamines, polylysines, PEG (monodisperse), albumin, fatty linear carbon chains, sugars (glycosylation), and most suitably are PEG (monodisperse).

By the term “cMet binding cyclic peptide” (cMBP) is meant a peptide which binds to the hepatocyte growth factor (HGF) high affinity receptor, also known as cMet (c-Met or hepatocyte growth factor receptor). Suitable cMBP peptides have an apparent Kd for cMet of cMet/HGF complex of less than about 10 μM. The cMBP peptides comprise proline residues, and it is known that such residues can exhibit cis/trans isomerisation of the backbone amide bond. The cMBP peptides described herein include any such isomers.

By the term “biocompatible cation” (B^{C}) is meant a positively charged counterion which forms a salt with an ionised, negatively charged group, where said positively charged counterion is also non-toxic and hence suitable for administration to the mammalian body, especially the human body. Examples of suitable biocompatible cations include: the alkaline metals sodium or potassium; the alkaline earth metals calcium and magnesium; and the ammonium ion. Typical biocompatible cations are sodium and potassium, typically sodium.

By the term “amino acid” is meant an L- or D-amino acid, amino acid analogue (e.g., naphthylalanine) or amino acid mimetic which may be naturally occurring or of purely synthetic origin, and may be optically pure, i.e. a single enantiomer and hence chiral, or a mixture of enantiomers. Conventional 3-letter or single letter abbreviations for amino acids are used herein. The amino acids used may be optically pure. By the term “amino acid mimetic” is meant synthetic analogues of naturally occurring amino acids which are isosteres, i.e. have been designed to mimic the steric and electronic structure of the natural compound. Such isosteres are well known to those skilled in the art and include but are not limited to depsipeptides, retro-inverso peptides, thioam ides, cycloalkanes or 1,5-disubstituted tetrazoles [see M. Goodman, Biopolymers, 24, 137, (1985)].

By the term “peptide” is meant a compound comprising two or more amino acids, as defined above, linked by a peptide bond (i.e. an amide bond linking the amine of one amino acid to the carboxyl of another). The term “peptide mimetic” or “mimetic” refers to biologically active compounds that mimic the biological activity of a peptide or a protein but are no longer peptidic in chemical nature, that is, they no longer contain any peptide bonds (that is, amide bonds between amino acids). Here, the term peptide mimetic is used in a broader sense to include molecules that are no longer completely peptidic in nature, such as pseudo-peptides, semi-peptides and peptoids.

By the term “chelating agent” (IM) is meant a substance whose certain atoms can form several bonds to a single metal ion or metal ion salt. A chelating agent is a multidentate ligand often consisting in a macrocycle rich in electron giving elements such as nitrogen or oxygen.

One of the roles of the linker group —(A)ₘ,— of Formula I may be to distance the IM from the active site of the cMBP peptide. This is particularly important when the compound is relatively bulky, so that interaction with the target protein is not impaired. This can be achieved by a combination of flexibility (e.g. simple alkyl chains), so that the bulky group has the freedom to position itself away from the active site and/or rigidity such as a cycloalkyl or aryl spacer which orientate the IM away from the active site. The nature of the linker group can also be used to modify the biodistribution of the compound. Thus, e.g., the introduction of ether groups in the linker will help to modify plasma protein binding. When —(A)ₘ— comprises a polyethyleneglycol (PEG) building block or a peptide chain of 1 to 10 amino acid residues, the linker group may function to modify the pharmacokinetics and blood clearance rates of the compound in vivo. Such “biomodifier” linker groups may alter the rate of the clearance of the compound from background tissue, such as muscle or liver, and/or from the blood, thus giving a better diagnostic image due to less background interference. A biomodifier linker group may also be used to favour a particular route of excretion, e.g., via the kidneys as opposed to via the liver.

By the term “sugar” is meant a mono-, di- or tri- saccharide. Suitable sugars include: glucose, galactose, maltose, mannose, and lactose. Optionally, the sugar may be functionalised to permit facile coupling to amino acids. Thus, e.g., a glucosamine derivative of an amino acid can be conjugated to other amino acids via peptide bonds. The glucosamine derivative of asparagine (commercially available from NovaBiochem) is one example of this:

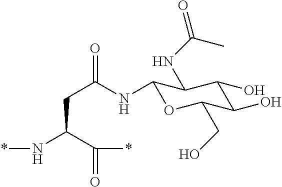 [SMILES: *N[C@@H](CC(=O)NC1OC(CO)C(O)C(O)C1NC(C)=O)C(*)=O]

The molecular weight of the imaging agent is suitably up to 8,000 Daltons. Optionally, the molecular weight is in the range 2,800 to 6,000 Daltons, typically 3,000 to 4,500 Daltons, with 3,200 to 4,000 Daltons being most typical.

Imaging agents of the present invention may have both peptide termini protected by M^{IG} groups, i.e. optionally both Z¹ and Z² are M^{IG}, which will usually be different. As noted above, either of Z¹/Z² may optionally equate to —(L)ₙ[IM]. Having both peptide termini protected in this way is important for in vivo imaging applications, since otherwise rapid metabolism would be expected with consequent loss of selective binding affinity for cMet. When both Z¹ and Z² are M^{IG}, optionally Z¹ is acetyl and Z² is a primary amide. Z¹ may be acetyl and Z² may be a primary amide and the —(L)ₙ[IM] moiety may be attached to the epsilon amine side chain of a lysine residue of cMBP.

cMBP peptides of the present invention may have a K_{D} for binding of cMet to cMet/HGF complex of less than about 10 nM (based on fluorescence polarisation assay measurements), most typically in the range 1 to 5 nM, with less than 3 nM being the ideal.

The peptide sequence (SEQ-1):

(SEQ-1)
Cys^{a}-Xaa¹-Cys^{c}-Xaa²-Gly-Pro-Pro-Xaa³-Phe-
Glu-Cys^{d}-Trp-Cys^{b}-Tyr-Xaa⁴-Xaa⁵-Xaa⁶

of the cMBP of Formula I is a 17-mer peptide sequence, which is primarily responsible for the selective binding to cMet. When the cMBP peptide of the present invention comprises more than 17 amino acid residues, the remaining amino acids can be any amino acid apart from cysteine. Additional, unprotected cysteine residues could cause unwanted scrambling of the defined Cys^{a}-Cys^{b} and Cys^{c}-Cys^{d} disulfide bridges. The additional peptides preferably comprise at least one amino acid residue with a side chain suitable for facile conjugation of the —(L)ₙ[IM] moiety. Suitable such residues include Asp or Glu residues for conjugation with amine-functionalised —(L)ₙ[IM] groups, or a Lys residue for conjugation with a carboxy- or active ester- functionalised —(L)ₙ[IM] group. The amino acid residues for conjugation of —(L)ₙ[IM] are suitably located away from the 17-mer binding region of the cMBP peptide (SEQ-1), and are optionally located at the C— or N— terminus. Optionally, the amino acid residue for conjugation is a Lys residue.

Substitution of the tryptophan residue of SEQ-1 was evaluated with the known amino acid substitutes phenylalanine and napthylalanine. Loss of cMet affinity was, however, found suggesting that the tryptophan residue is important for activity. Optionally the cMBP peptide further comprises a N-terminal serine residue, giving the 18-mer (SEQ-2):

(SEQ-2)
Ser-Cys^{a}-Xaa¹-Cys^{c}-Xaa²-Gly-Pro-Pro-Xaa³-
Phe-Glu-Cys^{d}-Trp-Cys^{b}-Tyr-Xaa⁴-Xaa⁵-Xaa⁶.

In addition to SEQ-1, or SEQ-2, the cMBP may further comprise either:
(i) an Asp or Glu residue, or an analogue thereof, within 4 amino acid residues of either the C— or N— peptide terminus of the cMBP peptide, and —(L)ₙ[IM] is functionalised with an amine group which is conjugated to the carboxyl side chain of said Asp or Glu residue, or analogue thereof, to give an amide bond; or
(ii) a Lys residue, or an analogue thereof, within 4 amino acid residues of either the C— or N— peptide terminus of the cMBP peptide, and -(L)ₙ[IM] is functionalised with a carboxyl group which is conjugated to the epsilon amine side chain of said Lys residue, or analogue thereof, to give an amide bond.

In addition to SEQ-1, or SEQ-2, the cMBP may further comprise a Lys residue, or an analogue thereof, within 4 amino acid residues of either the C— or N— peptide terminus of the cMBP peptide, and —(L)ₙ[IM] is functionalised with a carboxyl group which is conjugated to the epsilon amine side chain of said Lys residue, or analogue thereof, to give an amide bond.

Analogues of Asp and/or Glu may include one or more or 2-aminobutanedioic acid, 2-aminohexanedioic acid, 2-aminoheptanedioic acid, 2-aminooctanedioic acid, 2-aminononanedioic acid, 2-aminodecanedioic acid, 2-aminoundecanedioic acid, and 2-aminododecanedioic acid.

Analogues of Lys may include one or more of 2,3-diaminopropanoic acid, 2,4-diaminobutanoic acid, 2,5-diaminopentanoic acid, 2,7-diaminoheptanoic acid, 2,8-diaminooctanoic acid, 2,9-diaminononanoic acid, 2,10-diaminodecanoic acid, 2,11-diaminoundecanoic acid, 2,12-diaminododecanoic acid.

When a synthetic linker group (L) is present, it may comprise terminal functional groups which facilitate conjugation to [IM] and Z¹—[cMBP]—Z². When L comprises a peptide chain of 1 to 10 amino acid residues, the amino acid residues may be independently chosen from histidine, glycine, lysine, arginine, aspartic acid, glutamic acid or serine, optionally may be independently chosen from glycine, lysine, arginine, aspartic acid, glutamic acid or serine. Optionally, L may comprise a peptide chain of 1 to 5 amino acids. Optionally, L may comprise a peptide chain of 2 amino acids. Optionally, L may comprise a peptide chain of 3 amino acids. The amino acid residues may be independently chosen from histidine, glycine, lysine, arginine, aspartic acid, glutamic acid or serine, optionally may be independently chosen from glycine, lysine, arginine, aspartic acid, glutamic acid or serine. The amino acid residues may be glycine.

In the synthetic linker group of formula —(A)ₘ—, each A may be an amino acid and m may be an integer of value 1 to 5. Optionally, each A may be an amino acid and m may be 2. Optionally, each A may be an amino acid and m may be 3. The amino acid may be independently chosen from histidine, glycine, lysine, arginine, aspartic acid, glutamic acid or serine, optionally may be independently chosen from glycine, lysine, arginine, aspartic acid, glutamic acid or serine. The, or each, amino acid may be glycine.

When L comprises a PEG moiety, it may comprise units derived from oligomerisation of the monodisperse PEG-like structures of Formulae IA (17-amino-5-oxo-6-aza-3, 9, 12, 15-tetraoxaheptadecanoic acid) or IB:

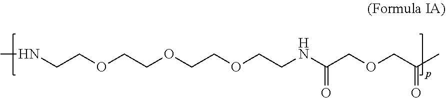

wherein p is an integer from 1 to 10. Alternatively, a PEG-like structure based on a propionic acid derivative of Formula IB can be used:

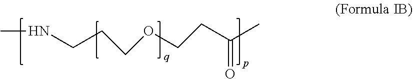

where p is as defined for Formula IA and q is an integer from 3 to 15. In Formula IB, p may be 1 or 2, and q may be 5 to 12.

L may comprise a peptide chain of 1 to 10 amino acid residues and a PEG moiety. In the synthetic linker group of formula —(A)ₘ—, each A may independently be an amino acid or a monodisperse polyethyleneglycol (PEG) building block. Optionally, each A may independently be an amino acid. m may be an integer of value 1 to 5, optionally 3, optionally 2.

When the linker group does not comprise PEG or a peptide chain, the L groups may have a backbone chain of linked atoms which make up the —(A)ₘ— moiety of 2 to 10 atoms, most preferably 2 to 5 atoms, with 2 or 3 atoms being especially preferred. A minimum linker group backbone chain of 2 atoms confers the advantage that the imaging moiety is well-separated so that any undesirable interaction is minimised. Peptides of formula Z¹—[cMBP]—Z² may be obtained by a method of preparation which comprises:
(i) solid phase peptide synthesis of a linear peptide which has the same peptide sequence as the desired cMBP peptide and in which the Cys^{a} and Cys^{b} are unprotected, and the Cys^{c} and Cys^{d} residues have thiol-protecting groups;
(ii) cleavage from the solid support and treatment of the peptide from step (i) with aqueous base in solution to give a monocyclic peptide with a first disulphide bond linking Cys^{a} and Cys^{b};
(iii) removal of the Cys^{c} and Cys^{d} thiol-protecting groups and cyclisation to give a second disulphide bond linking Cys^{c} and Cys^{d}, which is the desired bicyclic peptide product Z¹—[cMBP]—Z².

By the term “protecting group” is meant a group which inhibits or suppresses undesirable chemical reactions, but which is designed to be sufficiently reactive that it may be cleaved from the functional group in question under mild enough conditions that do not modify the rest of the molecule. After deprotection the desired product is obtained. Amine protecting groups are well known to those skilled in the art and are suitably chosen from: Boc (where Boc is tert-butyloxycarbonyl), Fmoc (where Fmoc is fluorenylmethoxycarbonyl), trifluoroacetyl, allyloxycarbonyl, Dde [i.e. 1-(4,4-dimethyl-2,6-dioxocyclohexylidene)ethyl] or Npys (i.e. 3-nitro-2-pyridine sulfenyl). Suitable thiol protecting groups are Trt (Trityl), Acm (acetamidomethyl), t-Bu (tert-butyl), tert-Butylthio, methoxybenzyl, methylbenzyl or Npys (3-nitro-2-pyridine sulfenyl). The use of further protecting groups is described in ‘Protective Groups in Organic Synthesis’, Theorodora W. Greene and Peter G. M. Wuts, (John Wiley & Sons, 1991). Typical amine protecting groups are Boc and Fmoc, most typically Boc. Other typical thiol protecting groups are Trt and Acm.

Further details of solid phase peptide synthesis are described in P. Lloyd-Williams, F. Albericio and E. Girald; Chemical Approaches to the Synthesis of Peptides and Proteins, CRC Press, 1997. The cMBP peptides are best stored under inert atmosphere and kept in a freezer. When used in solution, it is best to avoid pH above 7 since that risks scrambling of the disulfide bridges, and it is best to avoid low pH as this triggers aggregation of the peptide.

Z¹—[cMBP]—Z² may have both Z¹ and Z² equal to M^{IG}. Typical cMBP peptides and Z¹/Z² groups are as described above. In particular, it is typical that the cMBP peptide comprises an Asp, Glu or Lys residue to facilitate conjugation as described for the typical cMBP peptides described above. It is most typical that the cMBP peptide comprises a Lys residue.

The preparation of the Z¹—[cMBP]—Z² is described above. A Z¹—[cMBP]—Z³ peptide, where Z³ is an active ester, can be prepared from Z¹—[cMBP]—Z², where Z² is OH or a biocompatible cation (B^{C}), by conventional methods.

By the term “activated ester” or “active ester” is meant an ester derivative of the associated carboxylic acid which is designed to include a better leaving group, and hence permit more facile reaction with nucleophile, such as amines. Examples of suitable active esters are: N— hydroxysuccinimide (NHS), sulpho-succinimidyl ester, pentafluorophenol, pentafluorothiophenol, para-nitrophenol, hydroxybenzotriazole and PyBOP (i.e. benzotriazol-1-yl-oxytripyrrolidinophosphonium hexafluorophosphate). The active esters may be N-hydroxysuccinimide or pentafluorophenol esters, especially N-hydroxysuccinimide esters. Alternatively, an intramolecularly activated form of the carboxylic acid by formation of an anhydride may be used.

The radioactive moiety may be at least one of an alpha ray (a) emitter, a beta ray (β) emitter and a gamma ray (γ) emitter.

The beta ray emitter may be at least one of an electron (β⁻) emitter and a positron (β⁺) emitter.

The compound may be for use in one or more of positron-emission tomography (PET), single-photon emission computed tomography (SPECT), scintigraphy and radiotherapy. The compound may be for use in one or more of single-photon emission computed tomography (SPECT) and radiotherapy. The compound may be for use in radiotherapy.

The radioactive moiety may be selected from one or more of the group consisting of: ⁹⁰Y, ¹⁷⁷Lu, ¹⁸⁸Re, ⁶⁷Cu, ²¹²Bi, ²¹³Bi, ²¹¹At, ²²⁵Ac, ¹³¹I, ¹⁶⁶Ho, ¹⁴⁹Pm, ¹⁹⁹Au, ¹⁰⁵Rh, ²²⁷Th, ¹⁵³Sm, ⁸⁹Sr, ²²³Ra, ⁷⁷Br, ¹²³I, ¹²⁵I, ^{99m}Tc, ⁶⁷Ga, ¹¹¹In, ⁶⁸Ga, ⁶⁴Cu, ⁸⁹Zr, ¹¹C, ¹⁵O, ¹³N, ⁸²Rb and ¹⁸F, and suitable salts thereof.

The radioactive moiety may be selected from one or more of the group consisting of: ⁹⁰y, ¹⁷⁷Lu, ¹⁸⁸Re, ¹⁸⁶Re, ⁶⁷Cu, ²¹²Bi, ²¹³Bi, ²¹¹At, ²²⁵Ac, ¹³¹I, ¹⁶⁶Ho, ¹⁴⁹Pm, ¹⁹⁹Au, ¹⁰⁵Rh, ²²⁷Th, ¹⁵³Sm, ⁸⁹Sr, ²²³Ra, ⁷⁷Br, ¹²³I, and ¹²⁵I, and suitable salts thereof.

The radioactive moiety may be selected from one or more of the group consisting of: ⁶⁸Ga, ⁶⁴Cu, ⁸⁹Zr, ¹¹C, ¹⁵O, ¹³N, ⁸²Rb and ¹⁸F, and suitable salts thereof.

The radioactive moiety may be selected from one or more of the group consisting of: ^{99m}Tc, ⁶⁷Ga and ¹¹¹In, and suitable salts thereof.

The radioactive moiety may be selected from one or more of the group consisting of: ⁶⁸Ga, ¹⁸F, ⁸⁹Zr, ¹⁷⁷Lu, ²²⁵Ac, ²¹³Bi, ²²⁷Th and ⁹⁰Y, and suitable salts thereof.

The radioactive moiety may be ¹⁷⁷Lu, selected from one or more of the group consisting of: ⁶⁸Ga and ¹⁷⁷Lu, and suitable salts thereof.

The radioactive moiety may be ¹⁷⁷Lu, or suitable salts thereof.

The chelating agent may be selected from one or more of the group consisting of: cyclen (1,4,7,10-tetraazacyclododecane), cyclam (1,4,8,11-tetraazacyclotetradecane), TACN (1,4,7-triazacyclononane), THP (tris(hydroxypyridinone)), DOTAGA (2,2′,2″-(10-(1,4-dicarboxy-ethyl)-1,4,7,10-tetraazacyclododecane-1,4,7-triyl)triacetic acid), NODAGA (1,4,7-triazacyclononane,1-glutaric acid-4,7-acetic acid), TRAP (1,4,7-triazacyclononane phosphinic acid), NOPO (1,4,7-triazacyclononane-1,4-bis[methylene(hydroxymethyl)phosphinic acid]-72[methylene(2-carboxyethyl)phosphinic acid), NOTA (1,4,7-triazacyclononane-1,4,7-trisacetic acid), DOTA (1,4,7,10-tetraazacyclododecane-1,4,7, 10-tetraacetic acid), DATA ((6-pentanoic acid)-6-(amino)methy-1,4-diazepinetriacetate)), AAZTA (1,4-bis(carboxymethyl)-6-[bis(carboxymethyl)]amino-6-methylperhydro-1,4-diazepine), HBED-CC (N,N′-bis(2-hydroxy-5-(ethylene-beta-carboxy)benzyl)ethylenediamine N,N′-diacetic acid), and derivatives thereof.

The chelating agent may be at least one of: THP (tris(hydroxypyridinone)), DOTAGA (2,2′,2″-(10-(1,4-dicarboxy-ethyl)-1,4,7,10-tetraazacyclododecane-1,4,7-triyl)triacetic acid, DOTA (1,4,7,10-tetraazacyclododecane-1,4,7,10-tetraacetic acid), and NODAGA (1,4,7-triazacyclononane,1-glutaric acid-4,7-acetic acid).

The chelating agent may be at least one of: DOTAGA (2,2′,2″-(10-(1,4-dicarboxy-ethyl)-1,4,7,10-tetraazacyclododecane-1,4,7-triyl)triacetic acid), and DOTA (1,4,7,10-tetraazacyclododecane-1,4,7,10-tetraacetic acid).

The chelating agent may be DOTAGA (2,2′,2″-(10-(1,4-dicarboxy-ethyl)-1,4,7,10-tetraazacyclododecane-1,4,7-triyl)triacetic acid). The chelating agent may be DOTA (1,4,7,10-tetraazacyclododecane-1,4,7,10-tetraacetic acid).

In addition to SEQ-1, the cMBP further comprises an Asp or Glu residue within 4 amino acid residues of either C— or N— cMBP peptide terminus, and —(L)ₙIM is functionalised with an amine group, which is conjugated to the carboxyl side chain of said Asp or Glu residue to give an amide bond.

In addition to SEQ-1, the cMBP may comprise a Lys residue within 4 amino acid residues of either C— or N— cMBP peptide terminus, and —(L)ₙIM may be functionalised with a carboxyl group, which is conjugated to the epsilon amine side chain of said Lys residue to give an amide bond.

cMBP may comprise the amino acid sequence of either SEQ-2 or SEQ-3:

(SEQ-2)
Ser-Cys^{a}-Xaa¹-Cys^{c}-Xaa²-Gly-Pro-Pro-Xaa³-
Phe-Glu-Cys^{d}-Trp-Cys^{b}-Tyr-Xaa⁴-Xaa⁵-Xaa⁶;
(SEQ-3)
Ala-Gly-Ser-Cys^{a}-Xaa¹-Cys^{c}-Xaa²-Gly-Pro-
Pro-Xaa³-Phe-Glu-Cys^{d}-Trp-Cys^{b}-Tyr-Xaa⁴-
Xaa⁵-Xaa⁶-Gly-Thr.

Xaa³ may be Arg.

In addition to SEQ-1, SEQ-2 or SEQ-3, cMBP may further comprise at either the N— or C-terminus a linker peptide, which is chosen from-Gly-Gly-Gly-Lys (SEQ-4), -Gly-Ser-Gly-Lys- (SEQ-5) and -Gly-Ser-Gly-Ser-Lys (SEQ-6).

The Lys residue of the linker peptide is a typical location for conjugation of the —(L)ₙ[IM] moiety. Some cMBP peptides comprise SEQ-3 together with the linker peptide of SEQ-4, having the 26-mer amino acid sequence (SEQ-7):

(SEQ-7)
Ala-Gly-Ser-Cys^{a}-Tyr-Cys^{c}-Ser-Gly-Pro-Pro-
Arg-Phe-Glu-Cys^{d}-Trp-Cys^{b}-Tyr-Glu-Thr-Glu-
Gly-Thr-Gly-Gly-Gly-Lys.

cMBP may have the amino acid sequence (SEQ-7):

Ala-Gly-Ser-Cys^{a}-Tyr-Cys^{c}-Ser-Gly-Pro-Pro-
Arg-Phe-Glu-Cys^{d}-Trp-Cys^{b}-Tyr-Glu-Thr-Glu-
Gly-Thr-Gly-Gly-Gly-Lys.

Both Z¹ and Z² may independently be Q, optionally M.

Z¹ may be acetyl and Z² may be a primary amide.

n may be 1. n may be O.

cMBP peptides of SEQ-1, SEQ-2, SEQ-3 and SEQ-7 may have Z¹=Z²=M^{IG}, and may have Z¹=acetyl and Z²=primary amide.

The —(L)ₙ[IM] moiety is suitably attached to either of the Z¹ or Z² groups or an amino acid residue of the cMBP peptide which is different to the cMet binding sequence of SEQ-1. Possible amino acid residues and sites of conjugation are as described above. When the —(L)ₙ[IM] moiety is attached to Z¹ or Z², it may take the place of Z¹ or Z² by conjugation to the N— or C-terminus, and block in vivo metabolism in that way.

The compound may be suitable for the preparation of an agent for use in radiotherapy, the compound having Formula I:

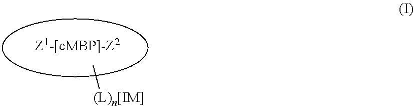

wherein:
Z¹ is attached to the N-terminus of cMBP, and is Q;
Z² is attached to the C-terminus of cMBP, and is Q;
   wherein Q is a metabolism inhibiting group (M^{IG}), which is a biocompatible group that inhibits or suppresses in vivo metabolism of the peptide;
cMBP is a cMet binding cyclic peptide of 17 to 30 amino acids, which comprises the amino acid sequence (SEQ-1):

Cys^{a}-Xaa¹-Cys^{c}-Xaa²-Gly-Pro-Pro-Xaa³-
Phe-Glu-Cys^{d}-Trp-Cys^{b}-Tyr-Xaa⁴-Xaa⁵-
Xaa⁶;

wherein: Xaa¹ is Asn, His or Tyr;
   Xaa² is Gly, Ser, Thr or Asn;
   Xaa³ is Thr or Arg;
   Xaa⁴ is Ala, Asp, Glu, Gly or Ser;
   Xaa⁵ is Ser or Thr;
   Xaa⁶ is Asp or Glu;
and Cys^{a-d} are each cysteine residues such that residues a and b as well as c and d are cyclised to form two separate disulphide bonds;
L is a synthetic linker group of formula —(A)ₘ— wherein each A is independently —CR₂—, —CR═CR—, —C═C—, —CR₂CO₂—, —CO₂CR₂—, —NRCO—, —CONR—, —NR(C═O)NR—, —NR(C═S)NR—, —SO₂NR—, —NRSO₂—, —CR₂OCR₂—, —CR₂SCR₂—, CR₂NRCR₂—, a C₄₋₈ cycloheteroalkylene group, a C₄₋₈ cycloalkylene group, a C₅₋₁₂ arylene group, a C₃₋₁₂ heteroarylene group, an amino acid, a sugar or a monodisperse polyethyleneglycol (PEG) building block;
each R is independently chosen from H, C₁₋₄ alkyl, C₂₋₄ alkenyl, C₂₋₄ alkynyl, C₁₋₄ alkoxyalkyl or C₁₋₄ hydroxyalkyl;
m is an integer of value 1 to 20;
n is an integer of value 0 or 1; and
IM is a chelating agent suitable for complexing a radioactive moiety,
   wherein the chelating agent is at least one of: DOTAGA (2,2′,2″-(10-(1,4-dicarboxy-ethyl)-1,4,7,10-tetraazacyclododecane-1,4,7-triyl)triacetic acid), and DOTA (1,4,7,10-tetraazacyclododecane-1,4,7,10-tetraacetic acid).

The compound may be suitable for the preparation of an agent for use in radiotherapy, the compound having Formula I:

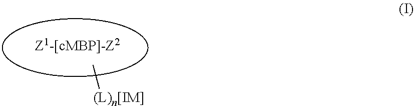

wherein:
Z¹ is attached to the N-terminus of cMBP, and is Q;
Z² is attached to the C-terminus of cMBP, and is Q;
   wherein Q is a metabolism inhibiting group (M^{IG}), which is a biocompatible group that inhibits or suppresses in vivo metabolism of the peptide;
cMBP is a cMet binding cyclic peptide of 17 to 30 amino acids, which comprises the amino acid sequence (SEQ-1):
Cys^{a}-Xaa¹-Cys^{c}-Xaa²-Gly-Pro-Pro-Xaa³-Phe-Glu-Cys^{d}-Trp-Cys^{b}-Tyr-Xaa⁴-Xaa⁵-Xaa⁶;
   wherein: Xaa¹ is Asn, His or Tyr;
      Xaa² is Gly, Ser, Thr or Asn;
      Xaa³ is Thr or Arg;
      Xaa⁴ is Ala, Asp, Glu, Gly or Ser;
      Xaa⁵ is Ser or Thr;
      Xaa⁶ is Asp or Glu;
   and Cys^{a-d} are each cysteine residues such that residues a and b as well as c and d are cyclised to form two separate disulphide bonds;
L is a synthetic linker group of formula —(A)ₘ— wherein each A is independently an amino acid or a monodisperse polyethyleneglycol (PEG) building block;
m is an integer of value 1 to 5;
n is 1; and
IM is a chelating agent suitable for complexing a radioactive moiety, wherein the chelating agent is at least one of: DOTAGA (2,2′,2″-(10-(1,4-dicarboxy-ethyl)-1,4,7,10-tetraazacyclododecane-1,4,7-triyl)triacetic acid), and DOTA (1,4,7,10-tetraazacyclododecane-1,4,7,10-tetraacetic acid).

According to a second aspect of the invention there is provided a pharmaceutical composition comprising the compound of the first aspect and a biocompatible carrier, in a form suitable for mammalian administration. The pharmaceutical composition may further comprise a radioactive moiety as described in the first aspect. The radioactive moiety can be complexed by the chelating agent.

The biocompatible carrier may be a solvent, typically an aqueous solvent, typically water. The solvent may be a fluid.

The “biocompatible carrier” may be a fluid, especially a liquid, in which the imaging agent can be suspended or dissolved, such that the composition is physiologically tolerable, i.e. can be administered to the mammalian body without toxicity or undue discomfort. The biocompatible carrier is suitably an injectable carrier liquid such as sterile, pyrogen-free water for injection; an aqueous solution such as saline (which may advantageously be balanced so that the final product for injection is isotonic); an aqueous solution of one or more tonicity-adjusting substances (e.g., salts of plasma cations with biocompatible counterions), sugars (e.g., glucose or sucrose), sugar alcohols (e.g., sorbitol or mannitol), glycols (e.g., glycerol), or other non-ionic polyol materials (e.g., polyethyleneglycols, propylene glycols and the like). The biocompatible carrier may be pyrogen-free water for injection or isotonic saline. The imaging agents and biocompatible carrier are each supplied in suitable vials or vessels which comprise a sealed container which permits maintenance of sterile integrity and/or radioactive safety, plus optionally an inert headspace gas (e.g., nitrogen or argon), whilst permitting addition and withdrawal of solutions by syringe or cannula. A preferred such container is a septum-sealed vial, wherein the gas-tight closure is crimped on with an overseal (typically of aluminium). The closure is suitable for single or multiple puncturing with a hypodermic needle (e.g., a crimped-on septum seal closure) whilst maintaining sterile integrity. Such containers have the additional advantage that the closure can withstand vacuum if desired (e.g., to change the headspace gas or degas solutions), and withstand pressure changes such as reductions in pressure without permitting ingress of external atmospheric gases, such as oxygen or water vapour.

The pharmaceutical composition may have a dosage for a single patient and may be provided in a suitable syringe or container.

According to a third aspect of the invention there is provided a kit for the preparation of the pharmaceutical composition of the second aspect, the kit comprising the compound of the first aspect in sterile, solid form such that upon reconstitution with a sterile supply of the biocompatible carrier of the second aspect, dissolution occurs to give the desired pharmaceutical composition.

The kit may further comprise a radioactive moiety as described in the first aspect. The radioactive moiety can be complexed by the chelating agent.

According to a further aspect of the invention there is provided a method of imaging of the mammalian body comprising use of at least one of the compound of the first aspect and the pharmaceutical composition of the second aspect.

The imaging may be in vivo.

The imaging may be at least one of PET imaging, scintigraphy and SPECT imaging.

The imaging may be SPECT imaging.

The imaging may be to obtain images of sites of cMet over-expression or localisation in vivo.

The imaging method, wherein optionally the compound of the first aspect or the pharmaceutical composition of the second aspect has been previously administered to the mammalian body.

The imaging method may comprise the steps of:
a) administering at least one of the compound of the first aspect and the pharmaceutical composition of the second aspect;
b) detecting emission from the radioactive moiety, which is generated by the decay of the radioactive moiety; and
c) forming an image of the tissue surface of interest from the emission of step (b).

The imaging method may be used to assist in detection, diagnosis, prognosis, prediction of outcome, surgery, staging, treatment, therapy, radiotherapy, monitoring of treatment, monitoring of disease progression and/or monitoring therapy.

The imaging method may be used to assist in detection, diagnosis, prognosis, prediction of outcome, surgery, staging, treatment, therapy, radiotherapy, monitoring of treatment, monitoring of disease progression and/or monitoring therapy of cancer or a pre-cancer condition.

A method of detection, diagnosis, prognosis, prediction of outcome, surgery, staging, treatment, therapy, radiotherapy, monitoring of treatment, monitoring of disease progression or monitoring therapy comprising the imaging method.

According to a further aspect of the invention there is provided the compound of the first aspect, or the pharmaceutical composition of the second aspect, for use as at least one of: an imaging agent in imaging of the mammalian body, and a radiotherapeutic agent in radiotherapy of the mammalian body.

According to a further aspect of the invention there is provided the compound of the first aspect, or the pharmaceutical composition of the second aspect, for use as a radiotherapeutic agent in radiotherapy of the mammalian body.

According to a further aspect of the invention there is provided the compound of the first aspect, or the pharmaceutical composition of the second aspect, for use as both: an imaging agent in imaging of the mammalian body, and a radiotherapeutic agent in radiotherapy of the mammalian body.

According to a further aspect of the invention there is provided at least one of the compound of the first aspect, and the pharmaceutical composition of claim the second aspect, for use as a medicament.

According to a further aspect of the invention there is provided at least one of the compound of the first aspect, and the pharmaceutical composition of the second aspect, for use in detection, diagnosis, prognosis, prediction of outcome, surgery, staging, treatment, therapy, radiotherapy, monitoring of treatment, monitoring of disease progression and/or monitoring therapy.

According to a further aspect of the invention, there is provided at least one of the compound of the first aspect, and the pharmaceutical composition of the second aspect, for use in the detection, diagnosis, prognosis, prediction of outcome, surgery, staging, treatment, therapy, radiotherapy, monitoring of treatment, monitoring of disease progression and/or monitoring therapy of cancer or a pre-cancer condition.

According to a further aspect of the invention there is provided at least one of the compound of the first aspect, and the pharmaceutical composition of the second aspect, for use in the detection, diagnosis, prognosis, prediction of outcome, surgery, staging, treatment, therapy, radiotherapy, monitoring of treatment, monitoring of disease progression and/or monitoring therapy of sites of cMet over-expression or localisation.

According to a further aspect of the invention there is provided at least one of the compound of the first aspect, and the pharmaceutical composition of the second aspect, for use in obtaining an image of, and/or treating conditions associated with, sites of cMet over-expression or localisation in vivo.

According to a further aspect of the invention there is provided a method of detection, diagnosis, prognosis, prediction of outcome, surgery, staging, treatment, therapy, radiotherapy, monitoring of treatment, monitoring of disease progression and/or monitoring therapy using at least one of the compound of the first aspect, and the pharmaceutical composition of the second aspect.

According to a further aspect of the invention there is provided a method of radiotherapy on the mammalian body using at least one of the compound of the first aspect, and the pharmaceutical composition of the second aspect.

According to a further aspect of the invention there is provided a method of both imaging and radiotherapy on the mammalian body using at least one of the compound of the first aspect, and the pharmaceutical composition of the second aspect.

According to a further aspect of the invention there is provided a method of detection, diagnosis, prognosis, prediction of outcome, surgery, staging, treatment, therapy, radiotherapy, monitoring of treatment, monitoring of disease progression and/or monitoring therapy of cancer or a pre-cancer condition, using at least one of the compound of the first aspect, and the pharmaceutical composition of the second aspect.

According to a further aspect of the invention there is provided a method of detection, diagnosis, prognosis, prediction of outcome, surgery, staging, treatment, therapy, radiotherapy, monitoring of treatment, monitoring of disease progression and/or monitoring therapy of sites of cMet over-expression or localisation using at least one of the compound of the first aspect, and the pharmaceutical composition of the second aspect.

According to a further aspect of the invention there is provided a method of obtaining an image of sites of and/or treating conditions associated with cMet over-expression or localisation in vivo using at least one of the compound of the first aspect, and the pharmaceutical composition of the second aspect.

According to a further aspect of the invention there is provided the use of at least one of the compound of the first aspect, and the pharmaceutical composition of the second aspect.

According to a further aspect of the invention there is provided the use of at least one of the compound of the first aspect, and the pharmaceutical composition of the second aspect, in at least one of detection, diagnosis, prognosis, prediction of outcome, surgery, staging, treatment, monitoring of treatment, therapy, radiotherapy, monitoring of disease progression and monitoring therapy.

According to a further aspect of the invention there is provided the use of at least one of the compound of the first aspect, and the pharmaceutical composition of the second aspect, as at least one of: an imaging agent, and a radiotherapeutic agent.

According to a further aspect of the invention there is provided the use of at least one of the compound of the first aspect, and the pharmaceutical composition of the second aspect, as a radiotherapeutic agent.

According to a further aspect of the invention there is provided the use of at least one of: the compound of the first aspect, and the pharmaceutical composition of the second aspect, in at least one of detection, diagnosis, prognosis, prediction of outcome, surgery, staging, treatment, therapy, radiotherapy, monitoring of treatment, monitoring of disease progression and monitoring therapy of cancer or a pre-cancer condition.

According to a further aspect of the invention there is provided the use of at least one of the compound of any the first aspect, and the pharmaceutical composition of the second aspect, in at least one of detection, diagnosis, prognosis, prediction of outcome, surgery, staging, treatment, therapy, radiotherapy, monitoring of treatment, monitoring of disease progression and monitoring therapy of sites of cMet over-expression or localisation.

According to a further aspect of the invention there is provided the use of at least one of the compound of the first aspect, and the pharmaceutical composition of the second aspect, in at least one of obtaining an image of, and treating conditions associated with, sites of cMet over-expression or localisation in vivo.

It will be appreciated that for in vivo use, the formulation is made into a form suitable for mammalian administration by, for example, reconstitution with a biocompatible carrier.

The alternative features and different embodiments as described apply to each and every aspect and each and every embodiment thereof mutatis mutandis.

### BRIEF DESCRIPTION OF THE DRAWINGS

Embodiments of the invention will now be described, by way of example, with reference to the drawings, in which:

FIG. 1 depicts a compound of the invention comprising NODAGA (Compound 1) and a reaction scheme to obtain Compound 1;

FIG. 2 depicts a compound of the invention comprising THP (Compound 2) and a reaction scheme to obtain Compound 2;

FIG. 3 depicts a compound of the invention comprising DOTA (Compound 3) and a reaction scheme to obtain Compound 3;

FIG. 4 depicts a compound of the invention comprising DOTAGA (Compound 4) and a reaction scheme to obtain Compound 4;

FIG. 5 shows in vivo PET/CT imaging of ⁶⁸Ga-Compound 3 (1 hour and 3 hours post injection) and ¹⁸F-FDG (1 hour post injection);

FIG. 6 shows in vivo SPECT imaging of ¹⁷⁷Lu-Compound 3 (1.5 hours, 17 hours, 41 hours, 65 hours and 141 hours post injection);

FIG. 7 shows in vivo PET/CT imaging of ¹⁸F-FDG (1 hour post injection); and

FIG. 8 shows in vivo SPECT imaging of ¹⁷⁷Lu-Compound 3 (4.5 hours, 20 hours, 45 hours, 67 hours and 141 hours post injection).

### DETAILED DESCRIPTION

Compounds useful as imaging or radiotherapy agents (or as precursors or as part of a kit) were prepared as described below.

Preparation of cMet Binding Peptide

Step (a): Synthesis of protected precursor linear peptide

The precursor linear peptide has the structure:

(comprising SEQ-7)
Ac-Ala-Gly-Ser-Cys-Tyr-Cys(Acm)-Ser-Gly-
Pro-Pro-Arg-Phe-Glu-Cys(Acm)-Trp-Cys-Tyr-
Glu-Thr-Glu-Gly-Thr-Gly-Gly-Gly-Lys-NH₂.

The peptidyl resin H-Ala-Gly-Ser(tBu)-Cys(Trt)-Tyr(tBu)-Cys(Acm)-Ser(tBu)-Gly-Pro-Pro-Arg(Pbf)-Phe-Glu(OtBu)-Cys(Acm)-Trp(Boc)-Cys(Trt)-Tyr(tBu)-Glu(OtBu)-Thr(Ψ^{Me}, ^{Me}pro)-Glu(OtBu)-Gly-Thr(tBu)-Gly-Gly-Gly-Lys(Boc)-Polymer (comprising SEQ-7) was assembled on an Applied Biosystems 433A peptide synthesizer using Fmoc chemistry starting with 0.1 mmol Rink Amide Novagel resin. An excess of 1 mmol pre-activated amino acids (using HBTU) was applied in the coupling steps. Glu-Thr pseudoproline (Novabiochem 05-20-1122) was incorporated in the sequence. The resin was transferred to a nitrogen bubbler apparatus and treated with a solution of acetic anhydride (1 mmol) and NMM (1 mmol) dissolved in DCM (5 mL) for 60 min. The anhydride solution was removed by filtration and the resin washed with DCM and dried under a stream of nitrogen.

The simultaneous removal of the side-chain protecting groups and cleavage of the peptide from the resin was carried out in TFA (10 mL) containing 2.5% TIS, 2.5% 4-thiocresol and 2.5% water for 2 hours and 30 min. The resin was removed by filtration, TFA removed in vacuo and diethyl ether added to the residue. The formed precipitate was washed with diethyl ether and air-dried affording 264 mg of crude peptide.

Purification by preparative HPLC (gradient: 20-30% B over 40 min where A=H₂O/0.1% TFA and B=ACN/0.1% TFA, flow rate: 10 mL/min, column: Phenomenex Luna 5μ C18 (2) 50×21.20 mm, detection: UV 214 nm, product retention time: 30 min) of the crude peptide afforded 100 mg of pure cMet Binding Peptide linear precursor. The pure product was analysed by analytical HPLC (gradient: 10-40% B over 10 min where A=H2CVO.1% TFA and B=ACN/0.1% TFA, flow rate: 0.3 mL/min, column: Phenomenex Luna 3 μ C18 (2) 50×2 mm, detection: UV 214 nm, product retention time: 6.54 min). Further product characterisation was carried out using electrospray mass spectrometry (MH₂²⁺calculated: 1464.6, MH₂²⁺found: 1465.1).

Step (b): Formation of Monocyclic Cys4-16 Disulfide Bridge

Cys4-16;
(comprising SEQ-7)
Ac-Ala-Gly-Ser-Cys-Tyr-Cys(Acm)-Ser-
Gly-Pro-Pro-Arg-Phe-Glu-Cys(Acm)-Trp-
Cys-Tyr-Glu-Thr-Glu-Gly-Thr-Gly-Gly-
Gly-Lys-NH₂.

The linear precursor from step (a) (100 mg) was dissolved in 5% DMSO/water (200 mL) and the solution adjusted to pH 6 using ammonia. The reaction mixture was stirred for 5 days. The solution was then adjusted to pH 2 using TFA and most of the solvent removed by evaporation in vacuo. The residue (40 mL) was injected in portions onto a preparative HPLC column for product purification.

Purification by preparative HPLC (gradient: 0% B for 10 min, then 0-40% B over 40 min where A=H₂O/0.1% TFA and B=ACN/0.1% TFA, flow rate: 10 mL/min, column: Phenomenex Luna 5μ C18 (2) 250×21.20 mm, detection: UV 214 nm, product retention time: 44 min) of the residue afforded 72 mg of pure cMet Binding Peptide monocyclic precursor. The pure product (as a mixture of isomers P1 to P3) was analysed by analytical HPLC (gradient: 10-40% B over 10 min where A=H₂O/0.1% TFA and B=ACN/0.1% TFA, flow rate: 0.3 mL/min, column: Phenomenex Luna 3μ C18 (2) 50×2 mm, detection: UV 214 nm, product retention time: 5.37 min (P1); 5.61 min (P2); 6.05 min (P3)). Further product characterisation was carried out using electrospray mass spectrometry (MH₂²⁺calculated: 1463.6, MH₂²⁺found: 1464.1 (P1); 1464.4 (P2); 1464.3 (P3)).

Step (c): Formation of Second Cys 6-14 Disulfide Bridge

The monocyclic precursor from step (b) (72 mg) was dissolved in 75% AcOH/water (72 mL) under a blanket of nitrogen. 1 M HCl (7.2 mL) and 0.05 M I₂ in AcOH (4.8 mL) were added in that order and the mixture stirred for 45 min. 1 M ascorbic acid (1 mL) was added giving a colourless mixture. Most of the solvents were evaporated in vacuo and the residue (18 mL) diluted with water/0.1% TFA (4 mL) and the product purified using preparative HPLC.

Purification by preparative HPLC (gradient: 0% B for 10 min, then 20-30% B over 40 min where A=H₂O/0.1% TFA and B=ACN/0.1% TFA, flow rate: 10 mL/min, column: Phenomenex Luna 5μ C18 (2) 250×21.20 mm, detection: UV 214 nm, product retention time: 43-53 min) of the residue afforded 52 mg of pure cMet Binding Peptide. The pure product was analysed by analytical HPLC (gradient: 10-40% B over 10 min where A=H₂O/0.1% TFA and B=ACN/0.1% TFA, flow rate: 0.3 mL/min, column: Phenomenex Luna 3μ C18 (2) 50×2 mm, detection: UV 214 nm, product retention time: 6.54 min). Further product characterisation was carried out using electrospray mass spectrometry (MH₂²⁺calculated: 1391.5, MH₂²⁺found: 1392.5).

Preparation of Compound 1

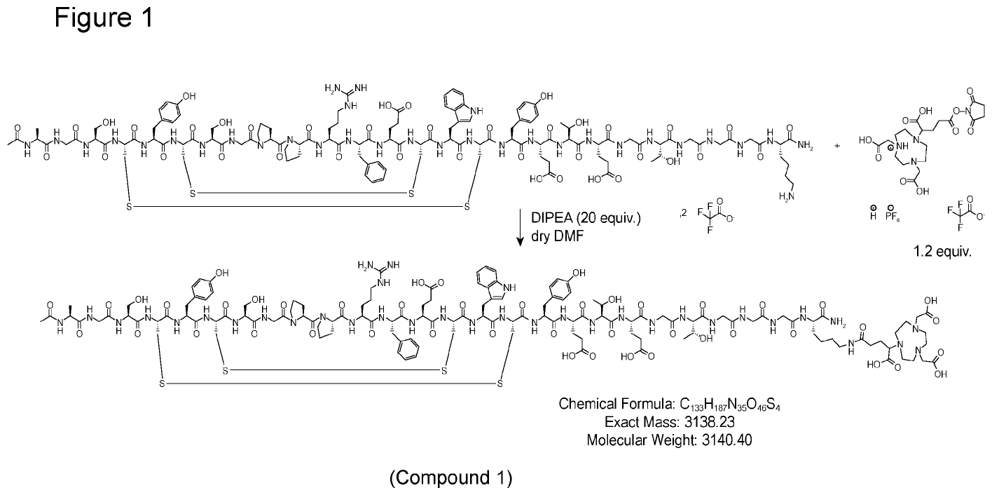

With reference to the reaction scheme at FIG. 1, Compound 1 was prepared as follows.

GMP grade cMet Binding Peptide (150.0 mg, 49.8 μmol) obtained from Bachem was dissolved in dry DMF (1.9 mL) in a 10 mL round bottomed flask under nitrogen. NODAGA-NHS ester (43.80 mg, 1.2 equivalents) obtained from CheMatec was dissolved in dry DMF (600 μL) and dry DIPEA (173 μL, 20.0 equivalents) obtained from Sigma Aldrich (99.5% Biotech grade) was added. The active ester solution was added to the peptide solution upon which a precipitate formed. The suspension was stirred for 1 hour at 25° C., at which point a sample was analysed by HPLC, showing incomplete reaction. The suspension was stirred for a further 1 hour, at which point a further sample was analysed by HPLC, again showing incomplete reaction. NODAGA-NHS ester (8.7 mg, 0.52 equivalents) and DIPEA (34.7 μL, 4 equivalents) were added to the reaction mixture. After 3 hours 30 minutes the reaction was stopped, even although conversion of the starting peptide to the product was incomplete (approx. 5% starting peptide remained). The reaction mixture was added ice cold MTBE (15 mL) and the precipitate was centrifuged (2 minutes, 3,260 rpm, 5° C.). The crude product was dissolved in 1 mL of water, which yielded a biphasic liquid, which was purified by semi-preparative HPLC (AA 100 mM, pH 7, AcN, 1% per minute, 10 mL per min, column Zorbax C18 30×250 mm, 3 injection, t_{R}=26.73 minutes). Compound 1 was obtained in moderate yield (76.9 mg, 49%) and excellent purity (>98%, 210 and 254 nm).

HPLC and MALDI/TOF confirmed that Compound 1 had been obtained.

HPLC Results

(Eluent A: 0.1% TFA in H₂O, Eluent B: 0.09% TFA in Acetonitrile; Column: Agilent Zorbax Eclipse Plus 95A C18, 2.1×150 mm, 3.5 um P/N 959763-902; Gradient: 5% B for 2.5 min, then 5% to 90% B in 85 min, then 90% B for 2.5 min, then 90% to 5% B in 2.5 min, then 5% B for 2.5 min; Detection: 210 nm)
t_{R} cMet peptide: 29.2 minutes; t_{R} product=30.1 minutes

(Eluent A: 0.1M ammonium acetate in water, Eluent B: Acetonitrile; Column: Agilent Zorbax Eclipse Plus 95A C18, 2.1×150 mm, 3.5 um P/N 959763-902; Gradient: 5% B for 2.5 min, then 5% to 90% B in 85 min, then 90% B for 2.5 min, then 90% to 5% B in 2.5 min, then 5% B for 2.5 min; Detection: 210 nm)
t_{R} cMet peptide: 21.0 minutes; t_{R} product=19.15 minutes

(Eluent A: 0.1% TFA in H₂O, Eluent B: 0.09% TFA in Acetonitrile; Column: Agilent Zorbax Eclipse Plus 95A C18, 2.1×150 mm, 3.5 um P/N 959763-902; Gradient: 5% B for 2.5 min, then 5% to 90% B in 17 min, then 90% B for 2.5 min, then 90% to 5% B in 2.5 min, then 5% B for 2.5 min; Detection: 210 nm)
t_{R} product=12.6 minutes

MALDI/TOF Results
Peptide (reanalysed): 2,786.98 (M+4H)
Product: 3,141 (calculated product mass=3,140.43)

Preparation of Compound 2

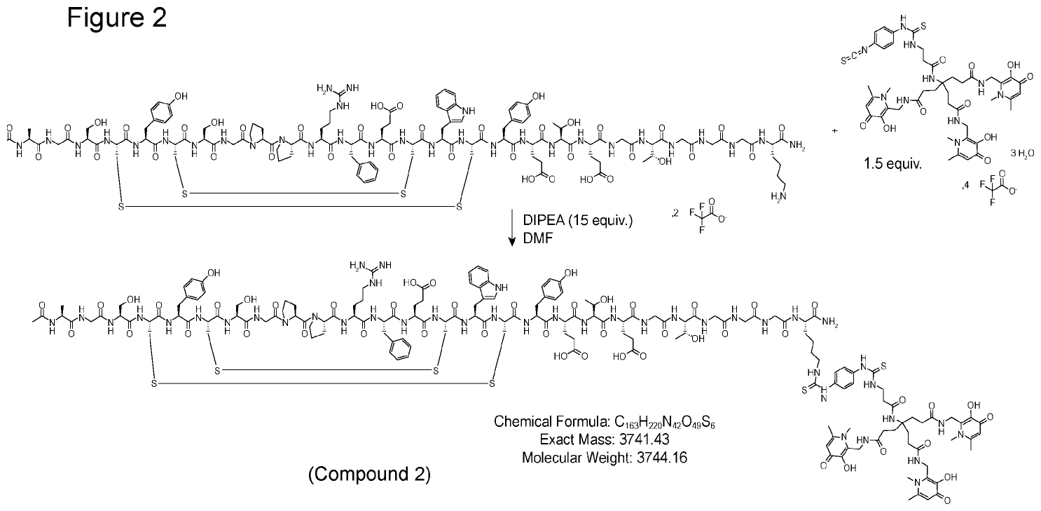

Compound 2 was prepared in accordance with the reaction scheme at FIG. 2, and as per the following description.

GMP grade c-Met peptide (30.0 mg, 9.97 μmol) and p-NCS-Bz-THP (14.37 mg, 1.5 equiv.) obtained from CheMatec were dissolved in dry DMF (1 mL) in a 10 mL round bottom flask. Dry DIPEA (26.0 μL, 15.0 equiv.) was added on which a precipitate formed. The partially solubilised suspension was stirred. HPLC analysis showed almost full conversion of the peptide after 2 hours and full conversion after 4 hours, by which time the solution is cloudy with some precipitate. To the reaction mixture was added ice cold MTBE (15 mL) and the precipitate was centrifuged (2 min, 3260 rpm, 5° C.). The washing was repeated with cold MTBE (6 mL) and the precipitate was centrifuged (2 min, 3260 rpm, 5° C.). The resulting white solid was dried under a flow of argon and then in vacuo (31.95 mg). The crude product was dissolved in 400 μL water and 400 μL AcN, and purified by semi preparative HPLC (TFA 0.1% in H₂O, AcN, 0.75% per minute, 20 mL per min, column Zorbax C18 21.2×250 mm, 1 injection, t_{R}=34.3 min, Trace file 521). The compound obtained in good yield and purity (89% UV 254 nm).

HPLC, ESI+/MS and MALDI/TOF confirmed that Compound 2 had been obtained.

HPLC Results

(Eluent A: 0.1% TFA in H₂O, Eluent B: 0.09% TFA in Acetonitrile; Column:

Agilent Zorbax Eclipse Plus 95A C18, 2.1×150 mm, 3.5 um P/N 959763-902; Gradient: 5% B for 2.5 min, then 5% to 90% B in 85 min, then 90% B for 2.5 min, then 90% to 5% B in 2.5 min, then 5% B for 2.5 min; Detection: 210 nm)
t_{R} product=13.7 minutes

ESI+/MS Results
Product: 1,872.8.3 (M+2H)²+; 1,248.5 (M+3H)³⁺

MALDI/TOF Results
Starting peptide: 2,786.98 (M+4H)⁺
Product: 3,738 (calculated product mass=3,741.43)

Preparation of Compound 3

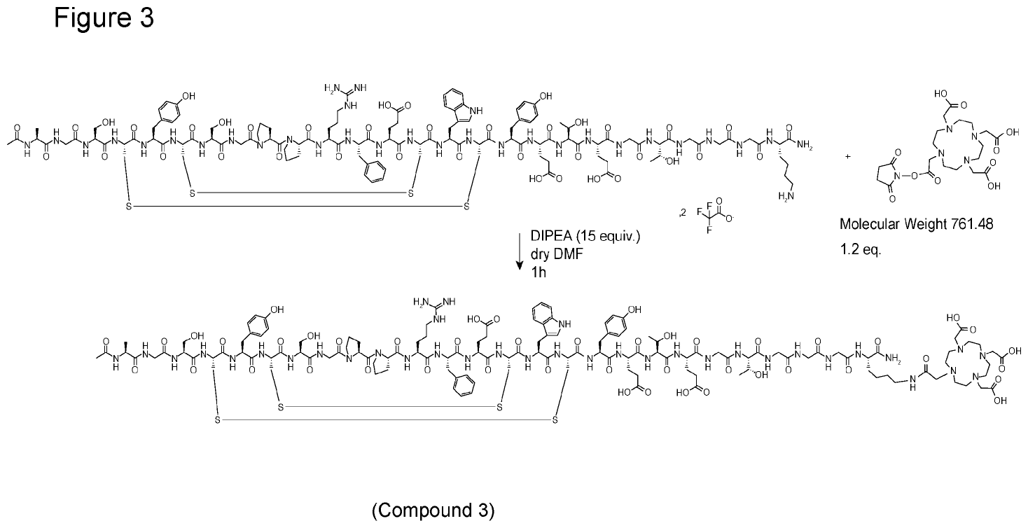

DOTA-NHS is commercially available as an activated species for the labelling of amines. It can be used for ⁶⁸Ga and ¹⁷⁷Lu chelation. DOTA-NHS was conjugated with the cMet Binding Peptide as per the reaction scheme in FIG. 3 to provide Compound 3. Further details are provided below.

GMP grade cMet Binding Peptide (10 mg, 3.17 μmol) obtained from Bachem was dissolved in dry DMF (100 μL) in a 1.5 mL Eppendorf tube and DIPEA (8.26 μL, 15.0 equivalents) obtained from Sigma Aldrich (99.5% Biotech grade) was added. DOTA-NHS (4.10 mg, 1.7 equivalents) obtained from CheMatec was dissolved in dry DMF (50 μL) and was added to the amine solution. The reaction mixture was flushed with Argon gas and reacted under positive pressure of Argon gas for approx. 24 hours at 20° C.

A sample was taken after 1 hour and was analysed by HPLC. A further sample was taken after approx. 24 hours and was analysed by ESI+/MS and MALDI/TOF to confirm complete reaction.

HPLC, ESI+/MS and MALDI/TOF all confirmed complete conversion of the cMet Binding Peptide to Compound 3.

HPLC Results (Eluent A: 0.1% TFA in H₂O, Eluent B: 0.09% TFA in Acetonitrile; Column: Agilent Zorbax Eclipse Plus 95A C18, 2.1×150 mm, 3.5 um P/N 959763-902; Gradient: 5% B for 2.5 min, then 5% to 90% B in 85 min, then 90% B for 2.5 min, then 90% to 5% B in 2.5 min, then 5% B for 2.5 min; Detection: 210 nm)
T₀=t_{R} cMet peptide: 29.2 minutes
T_{1H}=t_{R} cMet peptide: no peak; t_{R} product: 28.9 minutes

ESI+/MS Results
Reaction mixture after approx. 24 hours-positive ionisation:

1,585.3 (M+2H)²⁺; 1,056.9 (M+3H)³⁺

MALDI/TOF Results
Peptide (reanalysed): 2,785.05
Reaction mixture after approx. 24 hours: 3,170 (calculated product mass=3,169.44)

Preparation of Compound 4

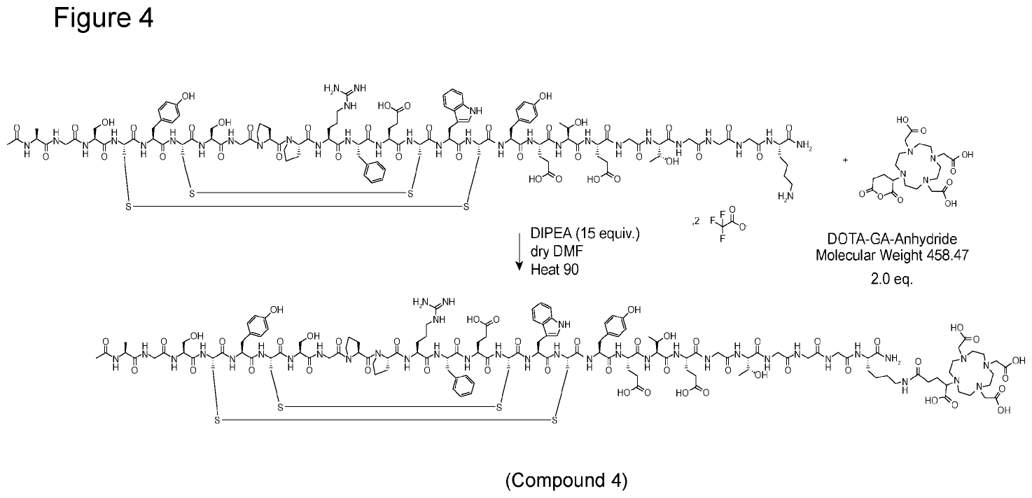

DOTAGA anhydride is commercially available as an activated species for the labelling of amines. It can be used for ⁶⁸Ga and ¹⁷⁷Lu chelation. DOTAGA anhydride was conjugated with the cMet Binding Peptide as per the reaction scheme in FIG. 4 to provide Compound 4. Further details are provided below.

GMP grade cMet Binding Peptide (25 mg, 7.92 μmol) obtained from Bachem was dissolved in dry DMF (300 μL) in a 10 mL round bottomed flask previously made moisture free by placing in an over at 180° C. DIPEA (21 μL, 15.0 equivalents) obtained from Sigma Aldrich (99.5% Biotech grade) was added to the round bottomed flask and flushed with Argon gas. DOTAGA anhydride (7.26 mg, 2.0 equivalents) obtained from CheMatec was dissolved in dry DMF (300 μL) in an Eppendorf tube and was added to the amine solution. The reaction mixture was sonicated to dissolve then flushed with Argon gas and reacted under positive pressure of Argon gas for approx. 4 hours at approx. 90° C.

Sample were taken after 30 minutes, 1 hour, 1 hour 30 minutes, 2 hours and four hours, and were analysed by HPLC. Each sample taken was analysed by MALDI/TOF and the sample taken at 4 hours was analysed by ESI+/MS to confirm complete reaction.

HPLC, ESI+/MS and MALDI/TOF all confirmed complete conversion of the cMet Binding Peptide to Compound 4.

HPLC Results (Eluent A: 0.1% TFA in H₂O, Eluent B: 0.09% TFA in Acetonitrile; Column: Agilent Zorbax Eclipse Plus 95A C18, 2.1×150 mm, 3.5 um P/N 959763-902; Gradient: 5% B for 2.5 min, then 5% to 90% B in 85 min, then 90% B for 2.5 min, then 90% to 5% B in 2.5 min, then 5% B for 2.5 min; Detection: 210 nm)
T_{30MIN}=t_{R} cMet peptide: 29.214 minutes; t_{R} product: 28.854 minutes
T_{1H}=t_{R} cMet peptide: 29.105 minutes; t_{R} product: 28.724 minutes
T_{1H3OMIN}=t_{R} cMet peptide: 29.112 minutes; t_{R} product: 28.745 minutes
T_{2H}=t_{R} cMet peptide: 29.607 minutes; t_{R} product: 28.796 minutes
T_{4H}=t_{R} cMet peptide: 29.560 minutes; t_{R} product: 28.747 minutes

ESI+/MS Results
Reaction mixture after approx. 4 hours-positive ionisation: 1,621.4

(M+2H)²+, 1,632.3 (M+Na+H)²⁺, 1,081.0 (M+3H)³⁺

MALDI/TOF Results
Reaction mixture after approx. 4 hours: 3,244.3 (M+H)⁺; 3266

(M+Na)⁺(calculated product mass=3,241.50)

The following experiments were performed on Compound 3 and Compound 4:
Residual Solvent Analysis (Experiment 1);
Determination of Counter Ion Stoichiometry (Experiment 2);
Competition Binding Assay by Fluorescence Polarisation (Experiment 3);
Radiolabelling Compound 3 and Compound 4 with ¹⁷⁷Lu (Experiment 4); and
Radiolabelling Compound 3 and Compound 4 with ⁶⁸Ga (Experiment 5).

Experiment 1: Residual Solvent Analysis of Compounds 3 and 4

Compounds 3 and 4 were tested for the following residual solvents: dimethylformamide (DMF); acetonitrile (MeCN); diisopropylethylamine (DIPEA); tert-butyl methyl ether (TBME); dichloromethane (DCM); and trifluoroacetic acid (TFA).

(a) Procedures

DMF, MeCN, DIPEA, TBME and DCM analysis was performed by Headspace Gas Chromatography (GC) as follows.

Samples of Compounds 3 and 4 were prepared, in duplicate, by accurately weighing out approximately 10 mg of each compound into separate HPLC headspace vials. 1 mL of dimethylamine (DMA) was added to each of the vials and the vials were sealed with crimp caps.

The prepared samples were analysed against a standard, which included the required solvents of interest.

The solvents in the standard were prepared at the working standard concentrations outlined in Table 1.

TABLE 1
Working Standard Concentrations

| | ICH | Den- | Vol- | Stock | Working |
|---|---|---|---|---|---|
| | Limit¹ | sity² | ume | Standard | Standard |
| Solvent | (ppm) | (g/mL) | (μL) | (μg/mL) | (μg/mL) |
| Acetonitrile | 410 | 0.786 | 100 | 3930 | 39 |
| Dichloromethane | 600 | 1.325 | 100 | 6625 | 66 |
| Tert-butyl methyl | 5000 | 0.740 | 600 | 22200 | 222 |
| ether | | | | | |
| Dimethylformamide | 880 | 0.944 | 200 | 9440 | 94 |
| Diisopropylethylamine | n/a | 0.742 | 200 | 7420 | 74 |

Note¹:
ICH Limit = International Council for Harmonisation of Technical Requirements for Pharmaceuticals for Human Use Limit.
Note²:
Density values taken from Sigma-Aldrich, all density values at 25° C.
n/a = not applicable

For analysis, 1 mL of standard was accurately pipetted into a 20 mL headspace vial and securely capped. One vial was prepared for each required injection of standard.

The samples and standards were analysed by GC on a Thermo Trace 1300 GC with Tri-Plus 300 Headspace Autosampler under the conditions outlined in Table 2.

TABLE 2
GC and Headspace Parameters
GC Parameters

| | |
|---|---|
| Column | Thermo TG0-624 30 m × 0.32 mm 1.8 μm |
| | film |
| Oven Temperature | 35° C. (hold 0.5 min) to 45° C. at 16.5° |
| | C./min to 70° C. at 5.0° C./min to 220° C. |
| | at 30.0° C./min |
| Flow Rate | 2.2 mL/min (constant flow) |
| Carrier Gas | Hydrogen |
| Injection Mode | Split |
| Injection Temperature | 225° C. |
| Injection Split Ratio | 20:1 |
| Detector Temperature | 250° C. |
| Detector Hydrogen | 40.0 mL/min |
| Detector Air | 400 mL/min |
| Make-up Flow | 30.0 mL/min |
| Make-up Gas | Nitrogen |

Headspace Parameters

| | |
|---|---|
| Oven Temperature | 100° C. |
| Manifold Temperature | 110° C. |
| Transfer Line Temperature | 150° C. |
| Vial Equilibration Time | 10.0 min |
| Pressurisation Mode | Pressure |
| Auxiliary Pressure | 100 kPa (Nitrogen) |
| Pressurisation Time | 0.2 min |
| Loop Fill Mode | Pressure |
| Loop Pressure | 50 kPa |
| Loop Fill Time | 0.2 min |
| Loop Equilibration Time | 0.05 min |
| Loop Volume | 1 mL |
| Inject Time | 0.5 min |
| Vial Shaking | High |
| GC Cycle Time | 21 min |

TFA analysis was performed by HPLC as follows.

A standard stock solution of TFA was prepared by pipetting 335 μL TFA into 100 mL of 0.008N sulfuric acid to give a 5 mg/mL stock. The stock solution was then diluted with 0.008N sulfuric acid to give standards at 0.1 mg/mL, 0.05 mg/mL, 0.025 mg/mL, 0.010 mg/mL and 0.005 mg/mL.

The standards were analysed by HPLC under the conditions outlined in Table 3. This produced a standard curve for quantitation of the amount of TFA in Compounds 3 and 4. The standard curve covered the range 500 to 10,000 ppm.

Samples of Compounds 3 and 4 were prepared, singly due to lack of availability of material, by accurately weighing out 20 mg of each compound into separate 2 mL volumetric flasks. The flasks were then made to volume with 0.008N sulfuric acid to provide 10 mg/mL solutions.

Both samples formed gels at 10 mg/mL, such that they were not suitable for injection onto the HPLC column. The samples were further diluted to 2 mg/m L. These samples were still viscous, but suitable for HPLC injection. The injection volume was increased to compensate for the dilution factor in the samples.

The HPLC conditions for TFA analysis are outlined in Table 3.

TABLE 3
HPLC Conditions for TFA Analysis

| | |
|---|---|
| Column | Bio-Rad Aminex HPX-87H 300 × 7.8 mm |
| Column Temperature | 35° C. |
| Mobile Phase | 0.008N Sulphuric Acid |
| Flow Rate | 0.6 mL/min |
| Run Time | 10 minutes |
| Injection Volume | 10 μL (standards), 50 μL (samples) |
| Detection | 210 nm |

(b) Results

The results obtained for the residual solvent determination by gas chromatography (GC) on Compounds 3 and 4 are shown in Table 4.

TABLE 4
Summary of Residual Solvent Analysis Results

| | DMF | MeCN | DIPEA | TBME | DCM |
|---|---|---|---|---|---|
| | ICH Limit¹ | ICH Limit¹ | ICH Limit¹ | ICH Limit¹ | ICH Limit¹ |
| | (ppm) | (ppm) | (ppm) | (ppm) | (ppm) |
| | 880 | 410 | no limit³ | 5000 | 600 |
| | Result | Result | Result | Result | Result |
| Compound | (ppm) | (ppm) | (ppm) | (ppm) | (ppm) |
| 3 | 516² | 59 | nd | nd | nd |
| 4 | 496² | 219 | nd | nd | nd |

Note:
¹ICH Limit = International Council for Harmonisation of Technical Requirements for Pharmaceuticals for Human Use Limit.
Note:
²DMF was detected as carryover in the blank injections by GC, and is reported as a maximum potential result, which is still below the ICH limit.
Note:
³DIPEA is not classified by the ICH Guideline Q3C (R5) for Residual Solvents, and as a result has no specified limit.
nd = not detected

TFA residual limits could not be determined by the proposed method due to dissociation of the TFA from the salt. Dissociation of TFA resulted in a TFA response from the samples that was far in excess of the anticipated level of <5000 ppm.

Therefore, the HPLC analysis was deemed unsuitable for determination of residual TFA in the samples.

(c) Conclusion

The residual solvents detected in Compounds 3 and 4 were within their respective International Council for Harmonisation of Technical

Requirements for Pharmaceuticals for Human Use (ICH) limits, which means that both Compound 3 and Compound 4 are suitable for human administration.

Experiment 2: Determination of Counter Ion Stoichiometry of Compounds 3 and 4

The trifluoroacetic acid (TFA) stoichiometry of Compounds 3 and 4 was determined by HPLC analysis.

(a) Procedure

Compounds 3 and 4 are both considered penta TFA salts. As such, the theoretical percentage of TFA in the compounds was calculated as follows:
Molecular weight of TFA=114.02 g/mol
Molecular weight of penta TFA salt=570.1 g/mol
Molecular weight of compound 3 (penta salt)=3739.6 g/mol
Molecular weight of compound 4 (penta salt)=3811.6 g/mol

Therefore, the theoretical percentage of TFA in each of the compounds is:

Compound 3=(570.1/3739.6)×100=15.24%TFA

Compound 4=(570.1/3811.6)×100=14.96%TFA

To determine stoichiometry, samples were prepared at 1 mg/mL and analysed against a TFA standard at 0.15 mg/mL, which was the intended counterion content (15%).

A TFA standard was prepared in duplicate at approximately 0.15 mg/mL by diluting 100 μL of TFA to 10 mL of 0.008N sulphuric acid to give a 15 mg/mL stock, which was then diluted (1 mL to 100 mL in 0.008N sulphuric acid) to give a 0.15 mg/mL standard solution.

Samples of Compounds 3 and 4 were prepared at approximately 1 mg/mL by weighing 5 mg of each compound into separate 5 mL volumetric flasks. The flasks were made to volume with 0.008N sulphuric acid.

The samples and standards were analysed by HPLC using the conditions outlined in Table 5.

TABLE 5
HPLC Conditions for TFA Analysis

| | |
|---|---|
| Column | Bio-Rad Aminex HPX-87H 300 × 7.8 mm |
| Column Temperature | 35° C. |
| Mobile Phase | 0.008N Sulphuric Acid |
| Flow Rate | 0.6 mL/min |
| Run Time | 10 minutes |
| Injection Volume | 10 μL (standards), 50 μL (samples) |
| Detection | 210 nm |

The recovery of the samples against the standards was calculated factoring in weights used and the dilutions. The recovery of the samples would determine the stoichiometry and the results would follow this pattern:
Penta salt=100% recovery
Tetra salt=80% recovery
Tris salt=60% recovery
Bis salt=40% recovery
Mono salt=20% recovery

(b) Results

The standard recovery was 107.0%. This was attributed to the standard preparation procedure that was followed. Specifically, pure TFA was difficult to handle due to its volatility. This caused problems in handling and measuring out the pure volatile material.

Compound 3 gave a recovery of 91.86%.

Compound 4 gave a recovery of 99.06%.

The recoveries were within normal confidence limits placed on a counterion determination.

The testing confirmed penta stoichiometry for Compound 3 and Compound 4 within 10% confidence limits.

Experiment 3: Competition Binding Assay of Compounds 3 and 4 by Fluorescence Polarisation (FP)

The half maximal inhibitory concentration (IC₅₀) and dissociation constant (K_{d}) values were determined by competition fluorescence polarisation (FP) assay for the binding of cMet binding cyclic peptide (unlabelled), cMet-DOTA (Compound 3) and cMet-DOTAGA (Compound 4) to cMet receptor.

The principle of the fluorescence polarisation method can briefly be described as follows:

Monochromatic light passes through a horizontal polarizing filter and excites fluorescent molecules in the sample. Only those molecules that are oriented properly in the vertically polarized plane adsorb light, become excited, and subsequently emit light. The emitted light is measured in both horizontal and vertical planes. The anisotropy value (A), is the ratio between the light intensities following the equation:

A=(Intensity with horizontal polarizer—Intensity with vertical polarizer)/(Intensity with horizontal polarizer+2* Intensity with vertical polarizer).

The fluorescence anisotropy measurements were performed in a 96-well flat bottom plate using a Molecular Devices M5 multi-mode plate reader.

The assay buffer used was phosphate-buffered saline (PBS), pH 7.4 with 0.01% Tween 20.

Stock Preparation

Fluorescent probe (cMet binding cyclic peptide with fluorescent label (Cy5)) was dissolved in assay buffer to give a 1 mM stock. A 1 μM parent stock and 50 nM working stock were also prepared.

cMet receptor (100 μg) was dissolved in assay buffer (1 mL) to give a parent stock of 775 nM. Working stocks of 500 nM and 167 nM were also prepared.

Peptides (cMet binding cyclic peptide (unlabelled), Compound 3 and Compound 4) were dissolved in assay buffer to give a parent stock of either 0.5 or 1 mM. Working stocks of 67 μM were also prepared.

Peptide Competition Assay

A 2-fold dilution screen of peptides (cMet binding cyclic peptide (unlabelled), Compound 3 and Compound 4; 30 μL/well of 67 μM stock) giving final concentrations of 20 μM to 20 nM was added to the plate. cMet receptor (30 μL/well of 167 μM stock) giving a final concentration of 50 nM was also added. Fluorescent probe (40 μL/well of 50 nM stock) giving a final concentration of 20 nM (100 μL total volume) was also added. Each well was repeated in triplicate. 100 μL assay buffer (in triplicate) was used as a non-fluorescent blank. The plate was incubated at 30° C. for 10 minutes before scanning. The plate was scanned in triplicate at ex/em/cut-off filter(COF) 640/675/665 nm at room temperature (25° C.).

The anisotropy was plotted against competing peptide concentration for each of cMet peptide (unlabelled), Compound 3 and Compound 4.

IC₅₀ values were determined by data fitting with the following equation using KaleidaGraph v4.03:

$$r = {r}_{0} + \frac{{r}_{1} - {r}_{0}}{1 + ( \frac{[ peptide ]}{{IC}_{50}} )}$$

where r is the observed anisotropy, r₀ is the anisotropy of the free probe, r₁ is the anisotropy of the fully bound probe, and [peptide] is the competing peptide concentration.

K_{d} values were determined by data fitting with the following equation using KaleidaGraph v4.03:

$$r = {r}_{0} + \frac{{r}_{1} - {r}_{0}}{1 + ( \frac{{K}_{dP} \cdot ( [ peptide ] + {K}_{d} )}{{K}_{d} \cdot [ cMet ]} )}$$

where r is the observed anisotropy, r₀ is the anisotropy of the free probe, r₁ is the anisotropy of the fully bound probe, [peptide] is the competing peptide concentration, K_{dp} is the dissociation constant for cMet receptor and the fluorescent probe, and K_{d} is the dissociation constant for the competing peptide binding to the cMet receptor.

The mean IC₅₀ values and K_{d} values are provided in Table 6 below.

TABLE 6
Mean IC₅₀ Values and K_{d} Values for cMet Binding Cyclic
Peptide (Unlabelled), Compound 3 and Compound 4

| Peptide | K_{d} (nM) | IC₅₀ (nM) |
|---|---|---|
| cMet peptide (unlabelled) | 6.9 ± 0.8 | 320 ± 37 |
| Compound 3 | 3.0 ± 0.5 | 139 ± 21 |
| Compound 4 | 2.6 ± 0.4 | 121 ± 18 |

The results showed that cMet binding cyclic peptide (unlabelled), Compound 3 and Compound 4 have comparable affinity for the cMet receptor and any variations in IC₅₀ and K_{d} values were well within experimental error.

Experiment 4: Radiolabelling Compound 3 and Compound 4 with ¹⁷⁷Lu

A study was carried out to determine the feasibility of radiolabelling Compounds 3 and 4 with Lutetium-177 (¹⁷⁷Lu).

Pilot Study

Various peptide (Compounds 3 and 4) to ¹⁷⁷Lu ratios were tested to evaluate the corresponding radiolabelling yields by HPLC and Instant Thin-Layer Chromatography (ITLC).

The radiolabelling procedure was performed using 0.4 M ammonium acetate at pH 5.6 (buffer 1 for tests 1 to 8) or 0.4 M ammonium acetate and 0.325 M gentisic acid at pH 4 (buffer 2 for tests 9 to 12). [¹⁷⁷Lu]LuCl₃ with a specific activity of >500 MBq/nmol was used for the radiolabelling studies. A specified volume (V_{177Lu} in Table 8) of [¹⁷⁷Lu]LuCl₃ was mixed with the appropriate buffer and 1 nmol of peptide (Compound 3 or Compound 4). The reaction mixture was incubated at 80° C. for an incubation time of 10, 20 or 30 minutes in a thermomixer system. A summary of the radiolabelling tests with various ratios of Compounds 3 and 4 to ¹⁷⁷Lu are outlined in Table 7. The specific conditions for each test are outlined in Table 8. At the end of incubation, 1 μL of the mixture was injected on the HPLC system and analysed using the conditions outlined in Table 9.

TABLE 7
Radiolabelling tests with various ratios
of Compounds 3 and 4 to ¹⁷⁷Lu

| | | Specific | | |
|---|---|---|---|---|
| Test | | Activity Targeted | n peptide | Activity ¹⁷⁷Lu |
| No. | Compound | (MBq/nmol) | (nmol) | (MBq) |
| 1 | 3 | 30 | 1 | 30 |
| 2 | 3 | 30 | 1 | 30 |
| 3 | 3 | 30 | 1 | 30 |
| 4 | 3 | 60 | 1 | 60 |
| 5 | 4 | 30 | 1 | 30 |
| 6 | 4 | 30 | 1 | 30 |
| 7 | 4 | 30 | 1 | 30 |
| 8 | 4 | 60 | 1 | 60 |
| 9 | 3 | 60 | 1 | 60 |
| 10 | 4 | 60 | 1 | 60 |
| 11 | 3 | 60 | 1 | 60 |
| 12 | 4 | 60 | 1 | 60 |

TABLE 8
Experimental conditions for each test outlined in Table 7

| | n | V | V | V | | Activity | Final | |
|---|---|---|---|---|---|---|---|---|
| Test | peptide | peptide | buffer 1 | buffer 2 | V_{177LU} | Loaded | V | |
| No. | (nmol) | (μL) | (μL) | (μL) | (μL) | (MBq) | (μL) | Incubation |
| 1 | 1 | 1 | 8.23 | — | 2.94 | 23.8 | 12.2 | 80° C., 10 m |
| 2 | 1 | 1 | 8.23 | — | 2.94 | 23.8 | 12.2 | 80° C., 20 m |
| 3 | 1 | 1 | 8.23 | — | 2.94 | 23.8 | 12.2 | 80° C., 30 m |
| 4 | 1 | 1 | 20.7 | — | 7.39 | 58.6 | 29.1 | 80° C., 10 m |
| 5 | 1 | 1 | 10.4 | — | 3.71 | 28.8 | 15.1 | 80° C., 10 m |
| 6 | 1 | 1 | 10.4 | — | 3.71 | 28.8 | 15.1 | 80° C., 20 m |
| 7 | 1 | 1 | 10.4 | — | 3.71 | 28.8 | 15.1 | 80° C., 30 m |
| 8 | 1 | 1 | 20.9 | — | 7.45 | 57.0 | 29.3 | 80° C., 10 m |
| 9 | 1 | 1 | — | 21 | 7.5 | 59.4 | 29.5 | 80° C., 10 m |
| 10 | 1 | 1 | — | 21 | 7.5 | 58.0 | 29.5 | 80° C., 10 m |
| 11 | 1 | 1 | — | 24.3 | 8.68 | 60.1 | 24.0 | 80° C., 10 m |
| 12 | 1 | 1 | — | 24.3 | 8.68 | 60.0 | 34.0 | 80° C., 10 m |

V = volume
m = minutes

TABLE 9
HPLC Conditions

| | |
|---|---|
| Column | Kinetex C18, 50 × 2.1 mm (Phenomenex) |
| Mobile Phase | Water (TFA 0.1%) and Acetonitrile (TFA 0.1%) |
| | Gradient (5% to 95% of Acetonitrile in 5 mins) |
| Flow Rate | 0.5 mL/min |
| Radioactivity | Bioscan module |
| Detection | |

The radiolabelling yield and radiochemical purity (RCP) was determined using HPLC and ITLC as follows. The radiolabelling yield defines the percentage of incorporation of ¹⁷⁷Lu before purification (i.e., the efficacy of radiolabelling).

Radiolabelling Yield by ITLC

The radiolabelling yield by ITLC was determined using the following equation:

$$Radiolabeling yield = \frac{Acitivity at Rf = 0}{Activity at Rf = 0 + Activity at Rf = 1} \times 100$$

where Rf is the retention factor, which is defined as the ratio of the distance travelled by a component in a sample to the distance travelled by the solvent front from the original point of application of the sample. Rf is 0 when the component remains at the point of application or origin. Rf is 1 when the component migrates with the solvent front.

Radiolabelling Yield and Radiochemical Purity by HPLC

The radiolabelling yield by HPLC was determined using the following equation:

$$Radiolabeling yield = \frac{Area Peak \#2 + Area Peak \#3 + Area Peak \#4}{Σ Areas Peak \#} \times 100$$

where area peak #2 is free ¹⁷⁷Lu, area peak #3 is by-product (or Compound 3 or 4 labelled with ¹⁷⁷Lu when there is no by-product peak) and area peak #4 is Compound 3 or 4 labelled with ¹⁷⁷Lu (when there is a by-product peak).

The radiochemical purity was determined using the following equation:

$$Radiolabeling purity = \frac{Area Peak \#3 ( or peak \#2 )}{Σ Areas Peak \#} \times 100$$

where area peak #2 is free ¹⁷⁷Lu and area peak #3 Compound 3 or 4 labelled with ¹⁷⁷Lu.

The results of the radiolabelling tests are provided in Table 10.

TABLE 10
Radiolabelling Yields, Radiochemical Purity and
Specific Activity Results for Tests 1 to 12.

| | Radiolabelling | Radiolabelling | Radiochemical | Final Specific |
|---|---|---|---|---|
| Test | Yield | Yield | Purity, RCP | Activity |
| No. | (HPLC) (%) | (ITLC) (%) | (HPLC) (%) | (MBq/nmol) |
| 1 | 99.2 | 99.9 | 98.0 | 23.3 |
| 2 | 98.8 | 99.9 | 96.8 | 23.0 |
| 3 | 98.6 | 99.9 | 96.3 | 22.9 |
| 4 | 99.0 | 99.9 | 96.4 | 56.5 |
| 5 | 98.6 | 99.8 | 95.8 | 27.6 |
| 6 | 97.3 | 99.8 | 91.7 | 26.4 |
| 7 | 96.5 | 99.9 | 85.2 | 24.5 |
| 8 | 98.0 | 99.9 | 94.5 | 53.9 |
| 9 | 99.2 | 99.9 | 98.8 | 58.7 |
| 10 | 99.0 | 99.9 | 98.3 | 59.3 |
| 11 | 99.1 | 99.9 | 99.1 | 59.6 |
| 12 | 98.9 | 99.9 | 98.9 | 59.3 |

Compounds 3 and 4 were both successfully labelled with ¹⁷⁷Lu at 30 and 60 MBq/nmol in 10 minutes at 80° C.

It was found that increasing the incubation time to 20 or 30 minutes resulted in poorer radiochemical purity. Buffer 1 and buffer 2 both enabled complete radiolabelling of Compounds 3 and 4.

The initial results showed >99% incorporation (radiolabelling yield) of the radionuclide and >99% radiochemical purity with Compounds 3 and 4 after 10 minutes incubation with ¹⁷⁷Lu.

Optimisation Study

Further tests were carried out to raise the specific activity to at least 120 MBq/nmol.

The radiolabelling procedure was performed using 0.4 M ammonium acetate and 0.325 M gentisic acid at pH 4 (buffer 2). ¹⁷⁷LuCl₃ with a specific activity of >500 MBq/nmol was used for the radiolabelling studies. A specified volume (V₁₇₇Lu in Table 12) of ¹⁷⁷LuCl₃ was mixed with buffer 2 and 1 nmol of peptide (Compound 3 or Compound 4). The reaction mixture was incubated at 80° C. for an incubation time of 10 minutes in a thermomixer system. A summary of the further radiolabelling tests with various ratios of Compounds 3 and 4 to ¹⁷⁷Lu are outlined in Table 11. The specific conditions for each test are outlined in Table 12. At the end of incubation, 1 μL of the mixture was injected on the HPLC system and analysed using the conditions outlined in Table 9.

TABLE 11
Further radiolabelling tests with various
ratios of Compounds 3 and 4 to ¹⁷⁷Lu

| | | Specific | | |
|---|---|---|---|---|
| Test | | Activity Targeted | n peptide | Activity ¹⁷⁷Lu |
| No. | Compound | (MBq/nmol) | (nmol) | (MBq) |
| 13 | 3 | 120 | 1 | 120 |
| 14 | 4 | 120 | 1 | 120 |
| 15 | 3 | 120 | 1 | 120 |
| 16 | 3 | 120 | 1 | 120 |
| 17 | 4 | 120 | 1 | 120 |
| 18 | 4 | 120 | 1 | 120 |
| 19 | 3 | 150 | 1 | 150 |
| 20 | 3 | 150 | 1 | 150 |
| 21 | 3 | 150 | 1 | 150 |
| 22 | 4 | 150 | 1 | 150 |
| 23 | 4 | 150 | 1 | 150 |
| 24 | 4 | 150 | 1 | 150 |

TABLE 12
Experimental conditions for each test outlined in Table 11

| | n | V | V | | | | |
|---|---|---|---|---|---|---|---|
| | pep- | pep- | buffer | | Activity | Final | |
| Test | tide | tide | 2 | V_{177LU} | Loaded | V | |
| No. | (nmol) | (μL) | (μL) | (μL) | (MBq) | (μL) | Incubation |
| 13 | 1 | 1 | 42.3 | 15.1 | 117.8 | 58.4 | 80° C., 10 m |
| 14 | 1 | 1 | 42.3 | 15.1 | 117.8 | 58.4 | 80° C., 10 m |
| 15 | 1 | 1 | 42.3 | 15.1 | 116.7 | 58.4 | 80° C., 10 m |
| 16 | 1 | 1 | 42.3 | 15.1 | 115.7 | 58.4 | 80° C., 10 m |
| 17 | 1 | 1 | 42.3 | 15.1 | 115.7 | 58.4 | 80° C., 10 m |
| 18 | 1 | 1 | 42.3 | 15.1 | 115.4 | 58.4 | 80° C., 10 m |
| 19 | 1 | 1 | 54.6 | 19.5 | 148.1 | 75.1 | 80° C., 10 m |
| 20 | 1 | 1 | 54.6 | 19.5 | 147.6 | 75.1 | 80° C., 10 m |
| 21 | 1 | 1 | 54.6 | 19.5 | 147.8 | 75.1 | 80° C., 10 m |
| 22 | 1 | 1 | 54.6 | 19.5 | 150.3 | 75.1 | 80° C., 10 m |
| 23 | 1 | 1 | 54.6 | 19.5 | 148.2 | 75.1 | 80° C., 10 m |
| 24 | 1 | 1 | 54.6 | 19.5 | 147.4 | 75.1 | 80° C., 10 m |

V = volume
m = minutes

The stability of the radiolabelled Compounds 3 and 4 (Compound 3-¹⁷⁷Lu radioconjugate and Compound 4-¹⁷⁷Lu radioconjugate) was also analysed by HPLC 24 hours after the initial radiolabelling.

The radiolabelling yield and radiochemical purity (RCP) were determined using HPLC and ITLC as defined above.

The results of the further radiolabelling tests are provided in Table 13.

TABLE 13
Radiolabelling Yields, Radiochemical Purity and
Specific Activity Results for Tests 13 to 24.

| | | | | Radio- | |
|---|---|---|---|---|---|
| | Radio- | Radio- | Radio- | chemical | |
| | labelling | labelling | chemical | Purity, RCP | Final |
| | Yield | Yield | Purity, RCP | at 24 hrs | Specific |
| Test | (HPLC) | (ITLC) | (HPLC) | (HPLC) | Activity |
| No. | (%) | (%) | (%) | (%) | (MBq/nmol) |
| 13 | 98.9 | 99.9 | 98.9 | 94.6 | 116.5 |
| 14 | 98.6 | 99.9 | 96.6 | 92.2 | 113.8 |
| 15 | 98.6 | 99.7 | 97.2 | 91.9 | 113.3 |
| 16 | 98.1 | 99.8 | 97.3 | 93.6 | 112.5 |
| 17 | 98.8 | 99.9 | 97.9 | 93.3 | 113.3 |
| 18 | 99.1 | 99.9 | 96.8 | 95.7 | 111.7 |
| 19 | 98.2 | 99.5 | 96.9 | 90.2 | 143.5 |
| 20 | 97.8 | 99.4 | 97.8 | 93.2 | 144.4 |
| 21 | 98.6 | 99.6 | 98.6 | 93.3 | 146.2 |
| 22 | 98.5 | 99.9 | 97.5 | 93.9 | 148.1 |
| 23 | 98.6 | 99.9 | 97.2 | 93.6 | 144.1 |
| 24 | 99.0 | 99.9 | 98.1 | 94.2 | 144.6 |

Compounds 3 and 4 were both successfully labelled with ¹⁷⁷Lu at 120 and 150 MBq/nmol at 80° C. for 10 minutes using 0.4 M ammonium acetate and 0.325 M gentisic acid at pH 4 as a buffer.

The radiochemical yields and radiochemical purities were >95%.

After 24 hours in solution, the HPLC results showed that the radiochemical purity was still >90%. The impurities observed after 24 hours may be due to radiolysis.

Clinical Study

A radiolabelling experiment in a clinical environment was also performed. In this experiment, the starting activity of ¹⁷⁷Lu was 7.6 GBq.

The radiolabelling experiment was performed as follows. Compound 3 (32 nmol) was dissolved in ultrapure water (2 μg/μL) and 1.5 mL of 0.4 M sodium acetate buffer at pH 4.8 (8 mg/mL). Gentisic acid was added and the mixture was transferred to a ¹⁷⁷Lu V-vial. The vial was heated for 25 minutes at 95° C. After dissolving the mixture with water for injection and subsequent sterile filtration, 6.9 GBq of Compound 3 conjugated to ¹⁷⁷Lu was achieved. A specific activity of >200MBq/nmol (n=3) was achieved. The radiochemical yield and radiochemical purity were both >99%.

The stability of the injection solution was also found to be very good with >98% radiochemical purity and <2% of two unknown impurities observed after 24 hours at room temperature.

Conclusion

The radiochemical purities and specific activities obtained mean that a relevant amount of radioactivity can be brought to the tumour (i.e., site of cMet over-expression) for targeted radiotherapy using this approach. A relevant amount of radioactivity is described as 2 to 8 GBq, typically 7.4

GBq. Furthermore, using the procedure described above means that large amounts of “cold” peptide (i.e., unlabelled peptide) are not required, which would saturate the cMet receptors and prevent any radioactivity being brought to the site of the tumour.

Experiment 5: Radiolabelling Compound 3 and Compound 4 with ⁶⁸Ga

Pilot Study

A pilot study was conducted to evaluate radiolabelling feasibility of Compounds 3 and 4 with ⁶⁸Ga. Radiolabelling yields and radiochemical purities (RCP) were determined by HPLC.

The radiolabelling procedure was performed using 1M sodium acetate buffer at pH 4.5. [⁶⁸Ga]GaCl₃ elution with cationic pre-purification was used for the radiolabelling studies. A specified volume (V_{68Ga} in Table 15) of [⁶⁸Ga]GaCl₃ was mixed with buffer, water, ethanol and 4 to 16 nmol of peptide (Compound 3 or Compound 4). The reaction mixture was incubated at 95° C. for an incubation time of 5 to 10 minutes in a thermomixer system. A summary of the radiolabelling tests with various ratios of Compounds 3 and 4 to ⁶⁸Ga are outlined in Table 14. The specific conditions for each test are outlined in Table 15. At the end of incubation, 2.5 μL of the mixture was injected on the HPLC system and analysed using the conditions outlined in Table 16.

TABLE 14
Radiolabelling tests with various
ratios of Compounds 3 and 4 to ⁶⁸Ga

| | | Specific | | |
|---|---|---|---|---|
| Test | | Activity Targeted | n peptide | Activity ⁶⁸Ga |
| No. | Compound | (MBq/nmol) | (nmol) | (MBq) |
| 1 | 3 | 15 | 4 | 60 |
| 2 | 4 | 15 | 4 | 60 |
| 3 | 3 | 15 | 16 | 240 |

TABLE 15
Experimental conditions for each test outlined in Table 14

| | n | V | V | V | V | | Activity | Final | |
|---|---|---|---|---|---|---|---|---|---|
| Test | peptide | peptide | buffer | water | ethanol | V_{68Ga} | Loaded | V | |
| No. | (nmol) | (μL) | (μL) | (μL) | (μL) | (μL) | (MBq) | (μL) | Incubation |
| 1 | 4 | 4 | 75 | 500 | 50 | 100 | 60 | 804 | 95° C., 5 m |
| 2 | 4 | 4 | 75 | 500 | 50 | 100 | 60 | 804 | 95° C., 5 m |
| 3 | 16 | 16 | 400 | 2000 | 200 | 100 | 120 | 3316 | 95° C., 5 m |
| | | | | | | | | | and 10 m |

V = volume
m = minutes

TABLE 16
HPLC Conditions

| | |
|---|---|
| Column | Kinetex C18, 50 × 2.1 mm (Phenomenex) |
| Mobile Phase | Water (TFA 0.1%) and Acetonitrile (TFA 0.1%) |
| | Gradient (5% to 95% of Acetonitrile in 5 mins) |
| Flow Rate | 0.5 mL/min |
| Radioactivity | Bioscan module |
| Detection | |

The radiolabelling yield and radiochemical purity (RCP) were determined using HPLC. The radiolabelling yield defines the percentage of incorporation of ⁶⁸Ga before purification (i.e., the efficacy of radiolabelling).

Radiolabelling Yield and Radiochemical Purity by HPLC

The radiolabelling yield by HPLC was determined using the following equation:

$$Radiolabeling yield = \frac{Area Peak \#2 + Area Peak \#3 + Area Peak \#4}{Σ Areas Peak \#} \times 100$$

where area peak #2 is free ⁶⁸Ga, area peak #3 is by-product (or Compound 3 or 4 labelled with ⁶⁸Ga when there is no by-product peak) and area peak #4 is Compound 3 or 4 labelled with ⁶⁸Ga (when there is a by-product peak).

The radiochemical purity was determined using the following equation:

$$Radiolabeling purity = \frac{Area Peak \#3 ( or peak \#2 )}{Σ Areas Peak \#} \times 100$$

where area peak #2 is free ⁶⁸Ga and area peak #3 Compound 3 or 4 labelled with ⁶⁸Ga.

The results of the radiolabelling tests are provided in Table 17.

TABLE 17
Radiolabelling Yield, Radiochemical Purity and
Specific Activity Results for Tests 1 to 3.

| | Radiolabelling | Radiochemical | Final Specific |
|---|---|---|---|
| Test | Yield | Purity, RCP | Activity |
| No. | (HPLC) (%) | (HPLC) (%) | (MBq/nmol) |
| 1 | 61.4 | 61.4 | 9.2 |
| 2 | 67 | 67 | 10.0 |
| 3-5 min | 56.6 | 56.6 | 8.5 |
| 3-10 min | 80.1 | 80.1 | 12.0 |

Compounds 3 and 4 were both successfully labelled with ⁶⁸Ga using the conditions outlined above. However, after purification, specific activity only reached I₂ MBq/nmol.

In the pilot study, approximately 60% radiochemical purity was obtained for both Compounds 3 and 4 after incubation with ⁶⁸Ga. No radiolysis was observed.

Repeat Pilot Study with Extended Labelling Time

Various peptide (Compounds 3 and 4) to ⁶⁸Ga ratios were tested to evaluate the corresponding radiolabelling yields by HPLC and Instant Thin-Layer Chromatography (ITLC).

The radiolabelling procedure was performed using 1M sodium acetate buffer at pH 4.5. ⁶⁸GaCl₃: fractionated elution was used for the radiolabelling studies. In a microtube a specified volume, V_{68Ga} in Table 19, (activity measured in the microtube approximately 7.3 MBq) of ⁶⁸GaCl₃ was mixed with (0.11×V_{68Ga}) of buffer and 0.24 to 1.36 nmol of peptide (Compound 3 or Compound 4). The reaction mixture was incubated at 95° C. for an incubation time of 10 minutes in a thermomixer system. A summary of the radiolabelling tests with various ratios of Compounds 3 and 4 to ⁶⁸Ga are outlined in Table 18. The specific conditions for each test are outlined in Table 19.

At the end of incubation, the microtube was centrifuged, and 2.5 μL of the mixture was injected on the HPLC system and analysed using the conditions outlined in Table 16.

For ITLC, the following procedures were followed:
1 μL of mixture was spotted on ITLC-SG paper and eluted with citrate buffer (0.1M, pH 5) as a mobile phase (ITLC 1); and
1 μL of mixture was spotted on ITLC-SG paper and eluted with a solution of ammonium acetate (5 M)/methanol 1/1 (ITLC 2).

TABLE 18
Radiolabelling tests with various
ratios of Compounds 3 and 4 to ⁶⁸Ga

| | | Specific | | |
|---|---|---|---|---|
| Test | | Activity Targeted | n peptide | Activity ⁶⁸Ga |
| No. | Compound | (MBq/nmol) | (nmol) | (MBq) |
| 4 | 3 | 30 | 0.24 | 7.7 |
| 5 | 3 | 20 | 0.36 | 7.7 |
| 6 | 3 | 10 | 0.73 | 7.7 |
| 7 | 3 | 5 | 1.46 | 7.7 |
| 8 | 4 | 30 | 0.24 | 7.7 |
| 9 | 4 | 20 | 0.36 | 7.7 |
| 10 | 4 | 10 | 0.73 | 7.7 |
| 11 | 4 | 5 | 1.46 | 7.7 |

TABLE 19
Experimental conditions for each test outlined in Table 18

| | n | V | | | | | |
|---|---|---|---|---|---|---|---|
| | pep- | pep- | V | | Activity | Final | |
| Test | tide | tide | buffer | V_{68Ga} | Loaded | V | |
| No. | (nmol) | (μL) | (μL) | (μL) | (MBq) | (μL) | Incubation |
| 4 | 0.24 | 2.4 | 11 | 100 | 7.7 | 113.43 | 95° C., 10 m |
| 5 | 0.36 | 3.6 | 11 | 100 | 7.7 | 114.65 | 95° C., 10 m |
| 6 | 0.73 | 7.3 | 11 | 100 | 7.7 | 118.30 | 95° C., 10 m |
| 7 | 1.46 | 14.6 | 11 | 100 | 7.7 | 125.60 | 95° C., 10 m |
| 8 | 0.24 | 2.4 | 11 | 100 | 7.7 | 113.43 | 95° C., 10 m |
| 9 | 0.36 | 3.6 | 11 | 100 | 7.7 | 114.65 | 95° C., 10 m |
| 10 | 0.73 | 7.3 | 11 | 100 | 7.7 | 118.30 | 95° C., 10 m |
| 11 | 1.46 | 14.6 | 11 | 100 | 7.7 | 125.60 | 95° C., 10 m |

V = volume
m = minutes

The radiolabelling yield and radiochemical purity (RCP) was determined using HPLC as described above. The radiolabelling yield by ITLC was determined as follows.

Radiolabelling Yield by ITLC

The radiolabelling yield by ITLC was determined using the following equation:

$$Radiolabeling yield = \frac{Acitivity at Rf = 0}{Activity at Rf = 0 + Activity at Rf = 1} \times 100$$

where Rf is the retention factor, which is defined as the ratio of the distance travelled by a component in a sample to the distance travelled by the solvent front from the original point of application of the sample. Rf is 0 when the component remains at the point of application or origin. Rf is 1 when the component migrates with the solvent front.

The results of the radiolabelling tests are provided in Table 20.

TABLE 20
Radiolabelling Yields, Radiochemical Purity and
Specific Activity Results for Tests 4 to 11.

| | Radio- | Radio- | Radio- | Radio- | |
|---|---|---|---|---|---|
| | labelling | chemical | labelling | labelling | Final |
| | Yield | Purity, RCP | Yield | Yield | Specific |
| Test | (HPLC) | (HPLC) | (ITLC 1) | (ITLC 2) | Activity |
| No. | (%) | (%) | (%) | (%) | (MBq/nmol) |
| 4 | 81.0 | 77.8 | 93.4 | 94.6 | 24.7 |
| 5 | 88.9 | 84.5 | 96.8 | 96.5 | 17.8 |
| 6 | 93.7 | 90.8 | 97.6 | 98.3 | 9.6 |
| 7 | 94.6 | 91.9 | 97.5 | 98.0 | 4.8 |
| 8 | 86.5 | 83.1 | 96.8 | 97.0 | 26.3 |
| 9 | 92.7 | 89.4 | 98.0 | 98.0 | 18.9 |
| 10 | 94.0 | 91.5 | 98.2 | 98.7 | 9.6 |
| 11 | 94.4 | 92.6 | 98.5 | 98.8 | 4.9 |

Compounds 3 and 4 were both successfully labelled with ⁶⁸Ga using the conditions outlined above. However, the radiolabelled Compounds 3 and 4 (Compound 3-⁶⁸Ga radioconjugate and Compound 4-⁶⁸Ga radioconjugate) were not pure enough to be suitable for intravenous administration to the human body. It was found that ethanol may be added to prevent radiolysis.

A specific activity of 26 MBq/nmol (test 8) was achieved with a radiochemical purity of approximately 83%.

Optimisation Study

Further tests were carried out to optimise ⁶⁸Ga incorporation into Compounds 3 and 4 based on the results of the pilot studies. Compound 3 was used for the optimisation study.

The radiolabelling procedure was performed using sodium acetate buffer, 1 M at pH 4.5 (buffer 1) or sodium acetate buffer, 0.5 M at pH 4.5 (buffer 2). ⁶⁸GaCl₃: fractionated elution was used for the radiolabelling studies.

Heating at 95° C.:

In a microtube a specified volume, V_{68Ga} in Table 22, (activity measured in the microtube approximately 7.3 MBq) of ⁶⁸GaCl₃ was mixed with (0.11×V_{68Ga}) of the appropriate buffer, 0.24 nmol of peptide (Compound 3 or Compound 4) and 11.3 μL of ethanol. The reaction mixture was incubated at 95° C. for an incubation time of 10 or 15 minutes in a thermomixer system.

Heating at 110° C.:

In a sealed glass v-vial a specified volume, V_{68Ga} in Table 22, (activity measured in the microtube approximately 29.2 MBq) of ⁶⁸GaCl₃ was mixed with (0.11×V_{68Ga}) of the appropriate buffer, 0.96 nmol of peptide (Compound 3 or Compound 4) and 45.4 μL of ethanol. The reaction mixture was incubated at 110° C. for an incubation time of 10 minutes in a heating block.

At the end of incubation, the microtube was centrifuged, and 2.5 to 5 μL of the mixture was injected on the HPLC system and analysed using the conditions outlined in Table 16.

A summary of the radiolabelling tests with various ratios of Compounds 3 and 4 to ⁶⁸Ga are outlined in Table 21. The specific conditions for each test are outlined in Table 22.

For ITLC, the following procedures were followed:
1 μL of mixture was spotted on ITLC-SG paper and eluted with citrate buffer (0.1M, pH 5) as a mobile phase (ITLC 1); and
1 μL of mixture was spotted on ITLC-SG paper and eluted with a solution of ammonium acetate (5 M)/methanol 1/1 (ITLC 2).

TABLE 21
Radiolabelling tests with various
ratios of Compounds 3 and 4 to ⁶⁸Ga

| | | Specific | | |
|---|---|---|---|---|
| Test | | Activity Targeted | n peptide | Activity ⁶⁸Ga |
| No. | Compound | (MBq/nmol) | (nmol) | (MBq) |
| 12 | 3 | 30 | 0.24 | 7.3 |
| 13 | 3 | 30 | 0.24 | 7.3 |
| 14 | 3 | 30 | 0.24 | 7.3 |
| 15 | 3 | 30 | 0.24 | 7.3 |
| 16 | 3 | 30 | 0.96 | 29.2 |
| 17 | 3 | 30 | 0.96 | 29.2 |
| 18 | 3 | 30 | 0.24 | 7.3 |
| 19 | 3 | 30 | 0.24 | 7.3 |
| 20 | 3 | 30 | 0.24 | 7.3 |
| 21 | 3 | 30 | 0.24 | 7.3 |
| 22 | 3 | 30 | 0.24 | 7.3 |
| 23 | 3 | 30 | 0.24 | 7.3 |

TABLE 22
Experimental conditions for each test outlined in Table 21

| | V | V | V | V | | | Activity | Final | |
|---|---|---|---|---|---|---|---|---|---|
| Test | peptide | buffer 1 | buffer 2 | EtOH | V_{68Ga} | | Loaded | V | |
| No. | (μL) | (μL) | (μL) | (μL) | (μL) | pH | (MBq) | (μL) | Incubation |
| 12 | 2.4 | 11 | — | 11.3 | 100 | <3.5 | 7.6 | 804 | 95° C., 10 m |
| 13 | 2.4 | 11 | — | 11.3 | 100 | <3.5 | 7.6 | 804 | 95° C., 15 m |
| 14 | 2.4 | 11 | — | 11.3 | 100 | <3.5 | 6.1 | 3316 | 95° C., 10 m |
| 15 | 2.4 | 11 | — | 11.3 | 100 | <3.5 | 6.1 | 804 | 95° C., 15 m |
| 16 | 9.7 | 44 | — | 45.37 | 400 | 3.6 | 31 | 804 | 110° C., 10 m |
| 17 | 9.7 | 44 | — | 45.37 | 400 | 3.6 | 23.7 | 3316 | 110° C., 10 m |
| 18 | 2.4 | — | 25 | — | 100 | 3.79 | 7.4 | 804 | 95° C., 10 m |
| 19 | 2.4 | — | 27 | — | 100 | 3.92 | 7.3 | 804 | 95° C., 10 m |
| 20 | 2.4 | — | 29 | — | 100 | 4.03 | 7.2 | 3316 | 95° C., 10 m |
| 21 | 2.4 | — | 25 | 10 | 100 | 3.93 | 7.1 | 804 | 95° C., 10 m |
| 22 | 2.4 | — | 27 | 10 | 100 | 4.04 | 7.2 | 804 | 95° C., 10 m |
| 23 | 2.4 | — | 29 | 10 | 100 | 4.14 | 7.1 | 3316 | 95° C., 10 m |

V = volume
m = minutes
EtOH = ethanol

The radiolabelling yield and radiochemical purity (RCP) were determined using HPLC and ITLC as defined above.

The results of the optimisation tests are provided in Table 23.

TABLE 23
Radiolabelling Yields, Radiochemical Purity and
Specific Activity Results for Tests 12 to 23.

| | Radio- | Radio- | Radio- | Radio- | | Final |
|---|---|---|---|---|---|---|
| | labelling | chemical | labelling | labelling | % | Specific |
| | Yield | Purity, RCP | Yield | Yield | ⁶⁸Ga | Activity |
| Test | (HPLC) | (HPLC) | (ITLC 1) | (ITLC 2) | col- | (MBq/ |
| No. | (%) | (%) | (%) | (%) | loids | nmol) |
| 12 | 71.7 | 71.7 | 89.8 | 84.8 | 5 | 22.5 |
| 13 | 65.5 | 65.5 | 86.4 | 82.2 | 4.2 | 20.6 |
| 14 | 64.1 | 62.9 | 88.5 | 84.6 | 3.9 | 15.6 |
| 15 | 77.8 | 75.9 | 93.9 | 88.8 | 5.1 | 18.8 |
| 16 | 44.1 | 44.1 | 73.8 | 68.9 | 4.9 | 14.1 |
| 17 | 34.2 | 33.0 | 73.7 | 62.7 | 11 | 7.3 |
| 18 | 85.2 | 82.9 | 96.6 | 94.7 | 1.9 | 25.1 |
| 19 | 10.2 | 10.2 | 41.6 | 20.7 | 20.9 | 3.1 |
| 20 | 81.5 | 78.6 | 94.5 | 94.8 | 0 | 23.4 |
| 21 | 5.5 | 5.5 | 14.9 | 7.6 | 7.3 | 1.6 |
| 22 | 3 | 3 | 16.4 | 5.8 | 10.6 | 0.9 |
| 23 | 0.9 | 0.9 | 12 | 2.6 | 9.4 | 0.3 |

The following conclusions were derived from the optimisation study:
increasing the incubation temperature to 110° C. resulted in lower radiolabelling yields (see tests 12 to 15 (95° C.) compared to tests 16 to 17 (110° C.));
a longer incubation time did not significantly increase the radiolabelling yield (see tests 12 and 14 (10 minutes) compared to tests 13 and 15 (15 minutes));
the addition of ethanol did not significantly prevent radiolysis (the results showed that radiolysis was never higher than 5% with or without the addition of ethanol);
the addition of ethanol appeared to increase the formation of ⁶⁸Ga colloids;
the pH of below 3.8 is required to ensure that an acceptable specific activity is reached; and
a purification step (preferably using a SEP-PAK C18 cartridge) prior to HPLC injection was required in order to reach aa suitable radiochemical purity.

During the optimisation study, a specific activity of up to 25 MBq/nmol (test 18) was achieved with a radiochemical purity of approximately 83%.

Conclusion

The radiochemical purities and specific activities obtained mean that a relevant amount of radioactivity can be brought to the tumour (i.e., site of cMet over-expression) for imaging using this approach. A relevant amount of radioactivity is described as approximately 12.5 to 75 MBq for experimental agents in the development stage.

The following further experiment was carried out using Compound 3:
In Vivo Dosimetry Study (Experiment 6).

Experiment 6: In Vivo Dosimetry Study of Compound 3

A dosimetry study was carried out using Compound 3 to investigate the suitability of the compounds described herein for use in imaging and radiotherapy.

(a) Patient Background

Details of the two human patients that took part in the study are provided in Table 24.

TABLE 24
Patient Details for In Vivo Study.

| | | Age | Weight | Height | |
|---|---|---|---|---|---|
| Patient | Sex | (years) | (kg) | (cm) | Primary Disease |
| 1 | Female | 56 | 55 | — | Adenocarcinoma of the |
| | | | | | Lung |
| 2 | Male | 62 | 75 | 187 | Adenocarcinoma of the |
| | | | | | Esophagastric Junction |
| | | | | | (AEG) |

(b) Procedures

Compound 3 was radiolabelled with ⁶⁸Ga to provide an imaging agent (⁶⁸Ga-Compound 3). The radiolabelling procedure used was as follows. [⁶⁸Ga]GaCl₃ was mixed with buffer (1M sodium acetate buffer at pH 4.5) and Compound 3. The reaction mixture was incubated at 95° C. for an incubation time of 10 minutes. A specific activity of 25 MBq/nmol was achieved with a radiochemical purity of approximately 98% without purification.

Compound 3 was radiolabelled with ¹⁷⁷Lu to provide a therapeutic agent (¹⁷⁷Lu-Compound 3). The radiolabelling procedure used was as follows. Compound 3 was dissolved in ultrapure water (2 μg/μl) and 1.5 mL of 0.4 M sodium acetate buffer at pH 4.8 (8 mg/mL). Gentisic acid was added and the mixture was transferred to a ¹⁷⁷Lu V-vial. The vial was heated for 25 minutes at 95° C. After dissolving the mixture with water for injection and subsequent sterile filtration, 6.9 GBq of ¹⁷⁷Lu-Compound 3 was achieved. A specific activity of >200MBq/nmol (n=3) was achieved.

Patient 1 was administered with the imaging agent (⁶⁸Ga-Compound 3) seven days before the therapeutic agent (¹⁷⁷Lu-Compound 3) was injected into the patient. For patient 1, the ⁶⁸Ga had an activity of 240 MBq and the ¹⁷⁷Lu had an activity of 835 MBq. Pre-therapeutic positron emission tomography/computed tomography (PET/CT) imaging was carried out 1 hour and 3 hours post injection with the imaging agent. One day prior to administration of the therapeutic agent, patient 1 was administered with ¹⁸F-fluorodeoxyglucose (¹⁸F-FDG). The ¹⁸F had an activity of 227 MBq. PET/CT imaging was carried out 1 hour post injection with ¹⁸F-FDG. Patient 1 was then administered with the therapeutic agent. Pre-therapeutic single-photon emission computed tomography (SPECT) imaging was then carried out 1.5 hours, 17 hours, 41 hours, 65 hours and 141 hours post injection with the therapeutic agent.

Patient 2 was administered with ¹⁸F-FDG four days before the therapeutic agent (¹⁷⁷Lu-Compound 3) was injected into the patient. For patient 2, the ¹⁸F had an activity of 208 MBq and the ¹⁷⁷Lu had an activity of 959 MBq. PET/CT imaging was carried out 1 hour post injection with ¹⁸F-FDG. Patient 2 was then administered with the therapeutic agent. Pre-therapeutic SPECT imaging was then carried out 4.5 hours, 20 hours, 45 hours, 67 hours and 141 hours post injection with the therapeutic agent.

For comparison, a third patient presenting with prostate cancer was administered with prostate-specific membrane antigen (PSMA) which is considered, at present, one of the most successful targets for imaging and therapy in nuclear medicine. The procedure used was as follows. The patient was administered with an imaging agent (PSMA labelled with ¹⁸F) nineteen days before a therapeutic agent (PSMA radiolabelled with ¹⁷⁷Lu) was injected into the patient. The ¹⁸F had an activity of 250 MBq and the ¹⁷⁷Lu had an activity of 9185 MBq. The activity of the ¹⁷⁷Lu-PSMA therapeutic agent was approximately ten times higher than the activity of the ¹⁷⁷Lu-Compound 3 therapeutic agents used in the dosimetry study. This was because dosimetry studies have already been completed for the PSMA agent and the higher dosage is considered safe for use in vivo. PET/CT imaging was carried out 1 hour post injection with ¹⁸F-PSMA. The patient was then administered with the ¹⁷⁷Lu-PSMA therapeutic agent. Pre-therapeutic SPECT imaging was then carried out 19 hours, 43 hours and 65 hours post injection with the therapeutic agent.

(c) Imaging

PET/CT imaging was performed using a Siemens Biograph mCT Flow PET/CT. SPECT imaging was performed using a Siemens Intevo SPECT/CT.

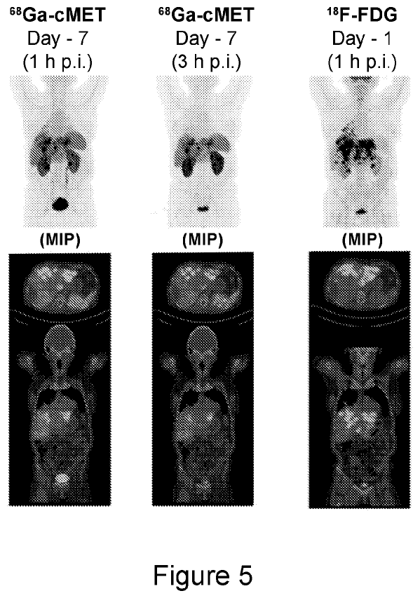

Pre-therapeutic PET/CT imaging results for patient 1 are shown in FIG. 5. The images show accumulation of ⁶⁸Ga-Compound 3 (referred to on the image as ⁶⁸Ga-cMET) in tumours at both 1 hour and 3 hours post injection (p.i.). ¹⁸F-FDG is also shown to accumulate in tumours 1 hour post injection.

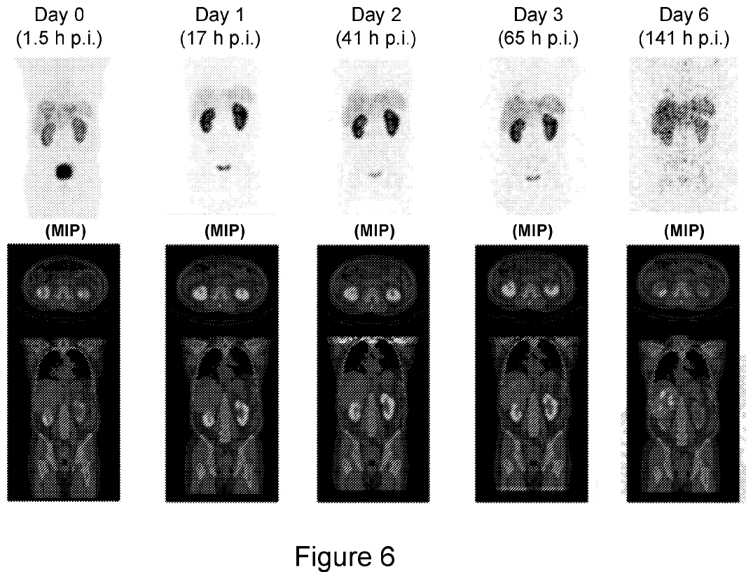

Pre-therapeutic SPECT imaging results for patient 1 are shown in FIG. 6. The image shows accumulation and retention of ¹⁷⁷Lu-Compound 3 in tumours up to 141 hours post injection (p.i.).

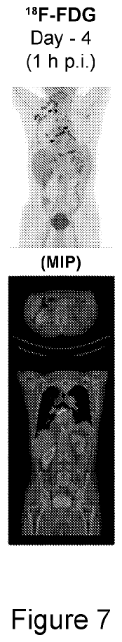

Pre-therapeutic PET/CT imaging results for patient 2 are shown in FIG. 7. The image shows accumulation of ¹⁸F-FDG in tumours 1 hour post injection.

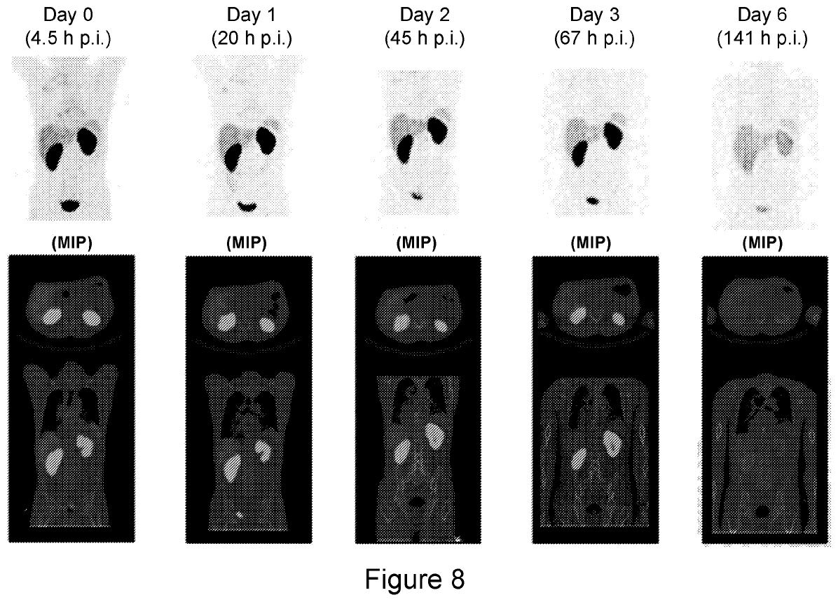

Pre-therapeutic SPECT imaging results for patient 2 are shown in FIG. 8. The image shows accumulation and retention of ¹⁷⁷Lu-Compound 3 in tumours up to 141 hours post injection (p.i.).

(d) Results

The results of the dosimetry study of the therapeutic agent (¹⁷⁷Lu-Compound 3) in patient 1 are provided in Table 25. The results of the dosimetry study of the therapeutic agent (¹⁷⁷-Lu-Compound 3) in patient 2 are provided in Table 26. For comparison, the results for the ¹⁷⁷Lu-PSMA therapeutic agent are provided in Table 27.

TABLE 25
Dosimetry Results of ¹⁷⁷Lu-Compound 3 in
Patient 1 (¹⁷⁷Lu Activity was 835 MBq).

| | | | | | | Mean |
|---|---|---|---|---|---|---|
| | | | | | Mean | Dose |
| | | | | | Dose | ((mGy) |
| | Cluster | Vol | Halflife | Dose | (Gy per | per (GBq |
| Tissue | Index | (ml) | (h) | (Gy) | GBq) | per kg)) |
| Kidney | 801 | 60 | 21 | 1.02 | 1.23 | 67.84 |
| (right) | | | | | | |
| Kidney | 800 | 84 | 21 | 1.04 | | |
| (left) | | | | | | |
| Spleen | 686 | 33 | 39 | 0.28 | 0.34 | 18.44 |
| Liver | 491 | 248 | 73 | 0.17 | 0.20 | 11.20 |
| Tumours | 754 | 6 | 42 | 0.4 | 0.45 | 24.70 |
| | 753 | 7 | 48 | 0.43 | | |
| | 756 | 4 | 32 | 0.36 | | |
| | 658 | 118 | 59 | 0.31 | | |

Vol = volume
h = hours

TABLE 26
Dosimetry Results of ¹⁷⁷Lu-Compound 3 in
Patient 2 (¹⁷⁷Lu Activity was 959 MBq).

| | | | | | | | Mean |
|---|---|---|---|---|---|---|---|
| | | | | | | Mean | Dose |
| | | | | Half- | | Dose | ((mGy) |
| | VOI | VOI | Vol | life | Dose | (Gy per | per (GBq |
| Tissue | Name | Index | (ml) | (h) | (Gy) | GBq) | per kg)) |
| Kidney | Not | 2548 | 135 | 40 | 1.06 | 1.61 | 128.06 |
| (right) | shown | | | | | | |
| | K-r-iso | Isocont. | 139 | — | 1.5 | | |
| Kidney | Not | 2466 | 135 | 37 | 1.87 | | |
| (left) | shown | | | | | | |
| | K-l-iso | Isocont. | 170 | — | 1.73 | | |
| Spleen | Spleen | 2368 | 118 | 58 | 0.13 | 0.14 | 10.20 |
| Liver | Liver | 2367 | 894 | 77 | 0.17 | 0.18 | 13.34 |
| Tumours | — | 2372 | 2 | 28 | 0.08 | 0.06 | 4.71 |
| | — | 2299 | 6 | 27 | 0.05 | | |
| | — | 2288 | 23 | 28 | 0.04 | | |

VOI = Volume of Interest
Vol = volume
h = hours

TABLE 27
Dosimetry Results of ¹⁷⁷Lu-PSMA therapeutic
agent (¹⁷⁷Lu Activity was 9185 MBq).

| | | | | | | Mean |
|---|---|---|---|---|---|---|
| | | | | | Mean | Dose |
| | | | | | Dose | ((mGy) |
| | VOI | Volume | Halflife | Dose | (Gy per | per (GBq |
| Tissue | Index | (ml) | (h) | (Gy) | GBq) | per kg)) |
| Kidney | 5742 | 71 | 36 | 2.6 | 0.28 | 26.37 |
| (right) | | | | | | |
| Kidney | 5741 | 85 | 33 | 2.5 | | |
| (left) | | | | | | |
| Spleen | 4745 | 237 | 17 | 0.3 | 0.03 | 3.10 |
| Liver | — | 268 | — | 0.3 | 0.03 | 3.10 |
| Tumours | 5894 | 37 | 57 | 22.8 | 1.89 | 179.11 |
| | 522 | 47 | 57 | 21.3 | | |
| | 491 | 156 | 58 | 10.8 | | |
| | 524 | 31 | 50 | 17.7 | | |
| | 527 | 19 | 47 | 15.5 | | |
| | 523 | 43 | 46 | 15.8 | | |

VOI = Volume of Interest
h = hours

The results show the accumulated dose of the therapeutic agents in the key tissues and organs.

Although the mean dose obtained in the tumours using ¹⁷⁷-Lu-Compound 3 was lower than that obtained using ¹⁷⁷Lu-PSMA, this was to be expected because of the lower activity of ¹⁷⁷Lu provided to the patients using Compound 3.

To evaluate the results, a literature search on dosimetry values for approved radiotherapeutic agents was conducted. ¹⁷⁷Lu-DOTATATE is an approved compound for systemic radiotherapy. Brogsitter et al. (Nuklearmedizin, 2017, Volume 56(1), pages 1-8) reported that ¹⁷⁷Lu-DOTATATE delivered a tumour dose of 5.53 Gy/GBq (median 2.70 Gy/GBq; range 0.44-15.3 Gy/GBq). Organ doses were reported to be as follows: kidney (2.03±0.96 Gy/GBq), liver (1.67±1.73 Gy/GBq), spleen (4.50±3.69 Gy/GBq) and whole body (0.15±0.08 Gy/GBq). The tumour-to-kidney dose ratio was reported as 2.4±5.6. Nicolas et al. (J Nucl Med, 2017, Volume 58, page 1435, doi: 10.2967/jnumed.117.191684) reported that ¹⁷⁷Lu-DOTATATE delivered a tumour dose of 0.333 Gy/GBq for a 4 cm tumour and had a tumour residence time of 6.4 hours (range 5.4-7.3 hours).

Furthermore, Okamoto et al. (J Nucl Med, 2016, Volume 116, doi: 10.2967/jnumed.116.178483) reported that ¹⁷⁷Lu-PSMA-I&T delivered tumour lesions with a mean absorbed dose per cycle of 3.2±2.6 Gy/GBq (range 0.22-I₂ Gy/GBq).

Typical limitations found during the literature search were either 23 Gy to the kidneys or 2 Gy to the bone marrow.

(e) Conclusion

The tumour doses delivered by ¹⁷⁷-Lu-Compound 3 are within the range of the approved radiotherapeutic agent ¹⁷⁷Lu-DOTATATE and late stage clinical development radiotherapeutic agent ¹⁷⁷Lu-PSMA. The tumour half-life was also found to be in a similar timeframe to ¹⁷⁷Lu-PSMA and longer than ¹⁷⁷Lu-DOTATATE. Additionally, organ doses to the kidney, liver and spleen using ¹⁷⁷-Lu-Compound 3 were all below the results reported for ¹⁷⁷Lu-DOTATATE.

Therefore, the dosimetry study shows that the compounds described herein are suitable for use in radiotherapy because the dose delivered to tumours is within the range observed for radiotherapeutic agents that have been approved or are in late stage of clinical development.

Various modifications and variations to the described embodiments of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes of carrying out the invention which are obvious to those skilled in the art are intended to be covered by the present invention.

### Abbreviations

Conventional single letter or 3-letter amino acid abbreviations are used.
AAZTA: 1,4-bis(carboxymethyl)-6-[bis(carboxymethyl)]amino-6-
methylperhydro-1,4-diazepine
Acm: Acetamidomethyl
ACN (or MeCN): Acetonitrile
Boc: tert-Butyloxycarbonyl
Bz: Benzoyl
Cyclam: 1,4,8,11-tetraazacyclotetradecane
Cyclen: 1,4,7,10-tetraazacyclododecane
CRC: Colorectal cancer
CT: Computed tomography
DATA: (6-pentanoic acid)-6-(amino)methy-1,4-diazepinetriacetate)
DCM: Dichloromethane
Dde: 1-(4,4-dimethyl-2,6-dioxocyclohexylidene)ethyl
DIPEA: N,N-diisopropylethylamine
DMA: Dimethylamine
DMF: Dimethylformamide
DMSO: Dimethylsulfoxide
DOTA: 1,4,7,10-tetraazacyclododecane-1,4,7,10-tetraacetic acid
DOTAGA: 2,2′,2″-(10-(1,4-dicarboxy-ethyl)-1,4,7,10-
tetraazacyclododecane-1,4,7-triyl)triacetic acid
ESI+/MS: Electrospray ionization mass spectrometry
EtOH: Ethanol
FDG: Fluorodeoxyglucose
FP: Fluorescence Polarisation
Fmoc: 9-Fluorenylmethoxycarbonyl
GC: Gas Chromatography
GBq: Gigabecquerel
GMP: Good manufacturing practices
HBED-CC: N,N′-bis(2-hydroxy-5-(ethylene-beta-
carboxy)benzyl)ethylenediamine N,N′-diacetic acid
HBTU: O-Benzotriazol-1-yl-N,N,N′,N-tetramethyluronium
hexafluorophosphate
HGF: Hepatocyte growth factor
HPLC: High performance liquid chromatography
HSPyU: O-(N-succinimidyl)-N, N, N′,N′-tetramethyleneuronium
hexafluorophosphate
IC₅₀: Half Maximal Inhibitory Concentration
ITLC: Instant Thin-Layer Chromatography
K_{d}: Dissociation Constant
MALDI/TOF: Matrix-assisted laser desorption ionization time-of-flight mass
spectrometry
MBq: Megabecquerel
MeCN: Acetonitrile
MTBE: Methyl tert-butyl ether
NCS: N-chloro-succinimide
NHS: N-hydroxy-succinimide
NMM: N-Methylmorpholine
NMP: 1-Methyl-2-pyrrolidinone
NODAGA: 1,4,7-triazacyclononane,1-glutaric acid-4,7-acetic acid
NOPO: 1,4,7-triazacyclononane-1,4-
bis[methylene(hydroxymethyl)phosphinic acid]-7-[methylene(2-
carboxyethyl)phosphinic acid
NOTA: 1,4,7-triazacyclononane-1,4,7-trisacetic acid
Npys: 3-nitro-2-pyridine sulfenyl
PEG: Polyethyleneglycol
PET: Positron emission tomography
Pbf: 2,2,4,6,7-Pentamethyldihydrobenzofuran-5-sulfonyl
PBS: Phosphate-buffered saline
PSMA: Prostate-specific membrane antigen
PyBOP: benzotriazol-1-yl-oxytripyrrolidinophosphonium
hexafluorophosphate
RCP: Radiochemical Purity
SPECT: Single-photon emission computed tomography
tBu: t-butyl
TACN: 1,4,7-triazacyclononane
TBME: tert-butyl methyl ether
TFA: Trifluoroacetic acid
THP: tris(hydroxypyridinone)
TIS: Triisopropylsilane
TRAP: 1,4,7-triazacyclononane phosphinic acid
Trt: Trityl

Sequence Listing Free Text
SEQ-1
Cys^{a}-Xaa¹-Cys^{c}-Xaa²-Gly-Pro-Pro-Xaa³-
Phe-Glu-Cys^{d}-Trp-Cys^{b}-Tyr-Xaa⁴-Xaa⁵-
Xaa⁶
Xaa¹ is Asn, His or Tyr;
Xaa² is Gly, Ser, Thr or Asn;
Xaa³ is Thr or Arg;
Xaa⁴ is Ala, Asp, Glu, Gly or Ser;
Xaa⁵ is Ser or Thr; and
Xaa⁶ is Asp or Glu.
17-mer cMET binding peptide.
SEQ-2
Ser-Cys^{a}-Xaa¹-Cys^{c}-Xaa²-Gly-Pro-Pro-
Xaa³-Phe-Glu-Cys^{d}-Trp-Cys^{b}-Tyr-Xaa⁴-
Xaa⁵-Xaa⁶
Xaa¹ is Asn, His or Tyr;
Xaa² is Gly, Ser, Thr or Asn;
Xaa³ is Thr or Arg;
Xaa⁴ is Ala, Asp, Glu, Gly or Ser;
Xaa⁵ is Ser or Thr; and
Xaa⁶ is Asp or Glu.
18-mer cMET binding peptide.
SEQ-3
Ala-Gly-Ser-Cys^{a}-Xaa¹-Cys^{c}-Xaa²-Gly-
Pro-Pro-Xaa³-Phe-Glu-Cys^{d}-Trp-
Cys^{b}-Tyr-Xaa⁴-Xaa⁵-Xaa⁶-Gly-Thr
Xaa¹ is Asn, His or Tyr;
Xaa² is Gly, Ser, Thr or Asn;
Xaa³ is Thr or Arg;
Xaa⁴ is Ala, Asp, Glu, Gly or Ser;
Xaa⁵ is Ser or Thr; and
Xaa⁶ is Asp or Glu.
22-mer cMET binding peptide.
SEQ-4
Gly-Gly-Gly-Lys
Tetrapeptide sequence that is part
of cMET binding peptide.
SEQ-5
Gly-Ser-Gly-Lys
Tetrapeptide sequence that is part
of cMET binding peptide.
SEQ-6
Gly-Ser-Gly-Ser-Lys
Five peptide sequence that is part
of cMET binding peptide.
SEQ-7
Ala-Gly-Ser-Cys-Tyr-Cys-Ser-Gly-Pro-
Pro-Arg-Phe-Glu-Cys-Trp-Cys-Tyr-Glu-
Thr-Glu-Gly-Thr-Gly-Gly-Gly-Lys
26-mer cMET binding peptide.

1-56. (canceled)

57. A compound suitable for the preparation of an agent for at least one of imaging and radiotherapy, the compound having Formula I: 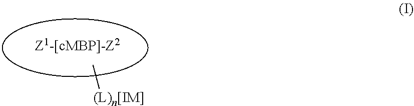
wherein:
Z¹ is attached to the N-terminus of cMBP, and is H or Q;
Z² is attached to the C-terminus of cMBP, and is OH, OB^{C} or Q;
wherein B^{C} is a biocompatible cation;
cMBP is a cMet binding cyclic peptide of 17 to 30 amino acids, which comprises the amino acid sequence (SEQ-1):
Cys^{a}-Xaa¹-Cys^{c}-Xaa²-Gly-Pro-Pro-Xaa³-Phe-Glu-Cys^{d}-Trp-Cys^{b}-Tyr-Xaa⁴-Xaa⁵-Xaa⁶;
wherein: Xaa¹ is Asn, His or Tyr;
Xaa² is Gly, Ser, Thr or Asn;
Xaa³ is Thr or Arg;
Xaa⁴ is Ala, Asp, Glu, Gly or Ser;
Xaa⁵ is Ser or Thr;
Xaa⁶ is Asp or Glu;
and Cys_{a-d} are each cysteine residues such that residues a and b as well as c and d are cyclised to form two separate disulphide bonds;
each occurrence of Q is independently at least one of:
a metabolism inhibiting group (M^{IG}), which is a biocompatible group that inhibits or suppresses in vivo metabolism of the peptide,
a tumour retention group (T^{RG}), which is a biocompatible group that enhances retention in tumour cells or the like in vivo, and
a biodistribution enhancement group (D^{EG}), which is a biocompatible group that enhances biodistribution and/or prolongs blood retention in vivo;
L is a synthetic linker group of formula —(A)ₘ- wherein each A is independently —CR₂—, —CR═CR—, —CR₂CO₂—, —CO₂CR₂—, —NRCO—, —CONR—, —NR(C═O)NR—, —NR(C═S)NR—, —SO₂NR—, —NRSO₂—, —CR₂OCR₂—, —CR₂SCR₂₋, CR₂NRCR₂₋, a C₄₋₈ cycloheteroalkylene group, a C₄₋₈ cycloalkylene group, a C₅₋₁₂ arylene group, a C₃₋₁₂ heteroarylene group, an amino acid, a sugar or a monodisperse polyethyleneglycol (PEG) building block;
each R is independently chosen from H, C₁₋₄ alkyl, C₂₋₄ alkenyl, C₂₋₄ alkynyl, C₁₋₄ alkoxyalkyl or C₁₋₄ hydroxyalkyl;
m is an integer of value 1 to 20;
n is an integer of value 0 or 1;
IM is a chelating agent suitable for complexing a radioactive moiety.

58. The compound of claim 57, wherein the radioactive moiety is at least one of an alpha ray (α) emitter, a beta ray (β) emitter and a gamma ray (γ) emitter.

59. The compound of claim 58, wherein the beta ray emitter is at least one of an electron (β⁻) emitter and a positron (β⁺) emitter.

60. The compound of claim 57, wherein the compound is for one or more of positron-emission tomography (PET), single-photon emission computed tomography (SPECT), scintigraphy and radiotherapy.

61. The compound of claim 57, wherein the radioactive moiety is selected from one or more of the group consisting of: ⁹⁰Y, ¹⁷⁷Lu, ¹⁸⁸Re, ¹⁸⁶Re, ⁶⁷Cu, ²¹²Bi, ²¹³Bi, ²¹¹At, ²²⁵Ac, ¹³¹I, ¹⁶⁶Ho, ¹⁴⁹Pm, ¹⁹⁹Au, ¹⁰⁵Rh, ²²⁷Th, ¹⁵³Sm, ⁸⁹Sr, ²²³Ra, ⁷⁷Br, ¹²³I, ¹²⁵I, ^{99m}Tc, ⁶⁷Ga, ¹¹¹In, ⁶⁸Ga, ⁶⁴Cu, ⁸⁹Zr, ¹¹C, ¹⁵O, ¹³N, ⁸²Rb and ¹⁸F, and suitable salts thereof.

62. The compound of claim 57, wherein the chelating agent is selected from one or more of the group consisting of: cyclen (1,4,7,10-tetraazacyclododecane), cyclam (1,4,8,11-tetraazacyclotetradecane), TACN (1,4,7-triazacyclononane), THP (tris(hydroxypyridinone)), DOTAGA (2,2′,2″-(10-(1,4-dicarboxy-ethyl)-1,4,7,10-tetraazacyclododecane-1,4,7-triyl)triacetic acid, NODAGA (1,4,7-triazacyclononane, l-glutaric acid-4,7-acetic acid), TRAP (1,4,7-triazacyclononane phosphinic acid), NOPO (1,4,7-triazacyclononane-1,4-bis[methylene(hydroxymethyl)phosphinic acid]-7-[methylene(2-carboxyethyl)phosphinic acid), NOTA (1,4,7-triazacyclononane-1,4,7-trisacetic acid), DOTA (1,4,7,10-tetraazacyclododecane-1,4,7,10-tetraacetic acid), DATA ((6-pentanoic acid)-6-(amino)methy-1,4-diazepinetriacetate)), AAZTA (1,4-bis(carboxymethyl)-6-[bis(carboxymethy)]amino-6-methylperhydro-1,4-diazepine), HBED-CC (N,N′-bis(2-hydroxy-5-(ethylene-beta-carboxy)benzyl)ethylenediamine N,N′-diacetic acid), and derivatives thereof.

63. The compound of claim 57, wherein the chelating agent is at least one of: THP (tris(hydroxypyridinone)), DOTAGA (2,2 ‘,2″-(10-(1,4-dicarboxy-ethyl)-1,4,7, 10-tetraazacyclododecane-1,4,7-triyl)triacetic acid), DOTA (1,4,7,10-tetraazacyclododecane-1,4,7,10-tetraacetic acid), and NODAGA (1,4,7-triazacyclononane,1-glutaric acid-4,7-acetic acid).

64. The compound of claim 57, wherein the chelating agent is at least one of: DOTAGA (2,2’,2″-(10-(1,4-dicarboxy-ethyl)-1,4,7,10-tetraazacyclododecane-1,4,7-triyl)triacetic acid), and DOTA (1,4,7, 10-tetraazacyclododecane-1,4,7, 10-tetraacetic acid).

65. The compound of claim 57, wherein in addition to SEQ-1, the cMBP further comprises an Asp or Glu residue within 4 amino acid residues of either C— or N— cMBP peptide terminus, and -(L)ₙIM is functionalised with an amine group, which is conjugated to the carboxyl side chain of said Asp or Glu residue to give an amide bond.

66. The compound of claim 57, wherein in addition to SEQ-1, the cMBP comprises a Lys residue within 4 amino acid residues of either C— or N— cMBP peptide terminus, and -(L)ₙIM is functionalised with a carboxyl group, which is conjugated to the epsilon amine side chain of said Lys residue to give an amide bond.

67. The compound of claim 57, wherein cMBP comprises the amino acid sequence of either SEQ-2 or SEQ-3:
(SEQ-2)
Ser-Cys^{a}-Xaa¹-Cys^{c}-Xaa²-Gly-Pro-Pro-
Xaa³-Phe-Glu-Cys^{d}-Trp-Cys^{b}-Tyr-Xaa⁴-
Xaa⁵-Xaa⁶;
(SEQ-3)
Ala-Gly-Ser-Cys^{a}-Xaa¹-Cys^{c}-Xaa²-Gly-
Pro-Pro-Xaa³-Phe-Glu-Cys^{d}-Trp-Cys^{b}-
Tyr-Xaa⁴-Xaa⁵-Xaa⁶-Gly-Thr.

68. The compound of claim 67, wherein in addition to SEQ-1, SEQ-2 or SEQ-3, cMBP further comprises at either the N— or C-terminus a linker peptide, which is chosen from -Gly-Gly-Gly-Lys (SEQ-4), -Gly-Ser-Gly-Lys- (SEQ-5) and -Gly-Ser-Gly-Ser-Lys (SEQ-6).

69. The compound of claim 57, wherein cMBP has the amino acid sequence of (SEQ-7):
Ala-Gly-Ser-Cys^{a}-Tyr-Cys^{c}-Ser-Gly-
Pro-Pro-Arg-Phe-Glu-Cys^{d}-Trp-Cys^{b}-
Tyr-Glu-Thr-Glu-Gly-Thr-Gly-Gly-
Gly-Lys.

70. The compound of claim 57, wherein both Z¹ and Z² are independently Q.

71. The compound of claim 70, wherein Q is M^{IG}.

72. The compound of claim 57, wherein Z¹ is acetyl and Z² is a primary amide.

73. The compound of claim 57, wherein each A may independently be an amino acid or a monodisperse polyethyleneglycol (PEG) building block.

74. The compound of claim 57, wherein m is an integer of value 1 to 5.

75. A compound suitable for the preparation of an agent for at least one of imaging and radiotherapy, the compound having Formula I: 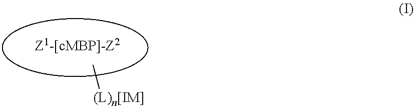
wherein:
Z¹ is attached to the N-terminus of cMBP, and is Q;
Z² is attached to the C-terminus of cMBP, and is Q;
wherein Q is a metabolism inhibiting group (M^{IG}), which is a biocompatible group that inhibits or suppresses in vivo metabolism of the peptide;
cMBP is a cMet binding cyclic peptide of 17 to 30 amino acids, which comprises the amino acid sequence (SEQ-1):
Cys^{a}-Xaa¹-Cys^{c}-Xaa²-Gly-Pro-Pro-Xaa³-
Phe-Glu-Cys^{d}-Trp-Cys^{b}-Tyr-Xaa⁴-Xaa⁵-
Xaa⁶;
wherein: Xaa¹ is Asn, His or Tyr;
Xaa² is Gly, Ser, Thr or Asn;
Xaa³ is Thr or Arg;
Xaa⁴ is Ala, Asp, Glu, Gly or Ser;
Xaa⁵ is Ser or Thr;
Xaa⁶ is Asp or Glu;
and Cys' are each cysteine residues such that residues a and b as well as c and d are cyclised to form two separate disulphide bonds;
L is a synthetic linker group of formula —(A)ₘ- wherein each A is independently an amino acid;
m is an integer of value 1 to 5;
n is 1; and
IM is a chelating agent suitable for complexing a radioactive moiety,
wherein the chelating agent is at least one of: DOTAGA (2,2′,2″-(10-(1,4-dicarboxy-ethyl)-1,4,7,10-tetraazacyclododecane-1,4,7-triyl)triacetic acid), and DOTA (1,4,7,10-tetraazacyclododecane-1,4,7,10-tetraacetic acid).

76. A pharmaceutical composition comprising the compound of claim 57 and a biocompatible carrier, in a form suitable for mammalian administration.

77. The pharmaceutical composition of claim 76, having a dosage for a single patient and being provided in a suitable syringe or container.

78. The pharmaceutical composition of claim 76, further comprising a radioactive moiety.

79. A kit for the preparation of the pharmaceutical composition of claim 76, the kit comprising the compound of claim 57 in sterile, solid form such that upon reconstitution with a sterile supply of the biocompatible carrier, dissolution occurs to give the desired pharmaceutical composition.

80. The kit of claim 79, further comprising a radioactive moiety.

81. A method of imaging a mammalian body comprising administering at least one of the compound of claim 57 and the pharmaceutical composition of claim 76.

82. The method of claim 81 comprising:
a) administering at least one of the compound of claim 57 and the pharmaceutical composition of claim 76;
b) detecting emission from the radioactive moiety, which is generated by the decay of the radioactive moiety; and
c) forming an image of a tissue of interest from the emission of step (b).

83. The method of claim 81, wherein the method is used to assist in at least one of detection, diagnosis, prognosis, prediction of outcome, surgery, staging, treatment, therapy, radiotherapy, monitoring of treatment, monitoring of disease progression and monitoring therapy of cancer or a pre-cancer condition.

84. The method of claim 81, wherein the method is used to assist in at least one of the detection, diagnosis, prognosis, prediction of outcome, surgery, staging, treatment, therapy, radiotherapy, monitoring of treatment, monitoring of disease progression and monitoring therapy of sites of cMet over-expression or localization.

85. The method of claim 81, wherein the method is used for at least one of obtaining an image of sites of cMet over-expression or localization in vivo and treating conditions associated with cMet over-expression or localization in vivo.

86. A method of radiotherapy on the mammalian body comprising administering at least one of the compound of claim 57 and the pharmaceutical composition of claim 76.

87. A method of both imaging and radiotherapy on the mammalian body comprising administering at least one of the compound of claim 57 and the pharmaceutical composition of claim 76.
